(19) United States
(12) Patent Application Publication
(10) Pub. No.: US 2014/0243300 A1
(43) Pub. Date: Aug. 28, 2014

(54) Controlling the Bioavailability of Active Ingredients in Topical Formulations

(71) Applicant: Precision Dermatology, Inc., Cumberland, RI (US)

(72) Inventors: Mark W. Trumbore, Westford, MA (US); Pinaki R. Majhi, Sharon, MA (US); Neha Aggarwal, Woonsocket, RI (US)

(73) Assignee: Precision Dermatology, Inc., Cumberland, RI (US)

(21) Appl. No.: 14/193,482

(22) Filed: Feb. 28, 2014

Related U.S. Application Data
(60) Provisional application No. 61/770,438, filed on Feb. 28, 2013.

(51) Int. Cl. A61K 47/46; A61K 31/573

(52) U.S. Cl. CPC A61K 47/46; A61K 31/573
USPC 514/179

(57) ABSTRACT

Described are methods and compositions for tuning the bioavailability of an active agent, such as a corticosteroid, in a topical formulation.

### RELATED APPLICATIONS

This application claims the benefit of priority to U.S. Provisional Patent Application Ser. No. 61/770,438, filed Feb. 28, 2013; the contents of which are hereby incorporated by reference.

### BACKGROUND

The bioavailability of a topically applied drug is strongly dependent upon the affinity of the drug for its vehicle. Vehicles with high affinities for a drug are associated with reduced rates of drug release and compromised bioavailability, which leads to poor therapeutic outcomes. Vehicles with low affinities for a drug are associated with elevated rates of drug release, excessive bioavailability, and increased potential for adverse events. Therefore, a need exists for methods to fine-tune drug release rates from any given topical formulation by varying the inactive constituents (e.g., vehicle) in order to maximize therapeutic outcomes while minimizing adverse events.

Mineral oils and vegetable oils are commonly used excipients in the oil phases of emulsion-based topical formulations. Although both classes of compounds are oils, their chemistries are fundamentally different. Vegetable oils are complex molecules with both hydrophilic and hydrophobic characteristics; in addition, they are heterodisperse (i.e., they comprise a range of individual fatty acids). In contrast, mineral oils, while still heterogeneous with respect to molecular structure, are much less complex; mineral oils are almost exclusively hydrophobic, and they primarily comprise alkyl chains.

Similarly, surfactants and co-surfactants are commonly used excipients in emulsion based topical formulations. They are used together to tailor emulsion droplet size and emulsion stability. Variation in co-surfactant/surfactant ratios is typically used to maximize formulation stability.

While oil-in-water emulsion-based topical formulations are known, the use of variations of oil phase composition to specifically engineer the rate of active ingredient release is not taught. For example, U.S. Pat. No. 5,635,497 teaches oil-in-water emulsion compositions in which the weight fraction of the discontinuous oil phase exceeds the weight fraction of the continuous water phase. However U.S. Pat. No. 5,635,497 does not teach the use of vegetable oils to increase the rate of active ingredient release and does not teach how the oil phase components and their ratios can be adjusted to maximize the ability of a topical composition to release incorporated active ingredients.

U.S. Pat. Nos. 7,378,405, 7,981,877, 8,399,502 and 8,546,364 teach oil-in-water emulsion formulations containing vegetable oils with high linoleic acid content. These patents teach the use of the vegetable oil as a chemical stabilizing agent for incorporated active ingredient and explicitly teach away from the use of vegetable oils containing low concentrations of linoleic acid. None of these patents teach the use of vegetable oils to increase the rate of active ingredient release or teach how the oil phase components and their ratios can be adjusted to maximize the ability of a topical composition to release incorporated active ingredients.

US patent application 2011/0305643 teaches oil-in-water emulsion-based aerosol foam compositions containing high weight percentages of oil phases. Although the compositions disclosed in 2011/0305643 contain vegetable oils, 2011/0305643 does not teach the use of vegetable oils to increase the rate of active ingredient release nor does it teach how the oil phase components and their ratios can be adjusted to maximize the ability of a topical composition to release incorporated active ingredients.

There exists a need for a method of tailoring of the release rate of active ingredients from topical formulations, thereby allowing precise control of topical bioavailability.

### SUMMARY OF THE INVENTION

In certain embodiments, the invention relates to a method of optimizing the rate of release of an active agent from a topical formulation, comprising the step of varying the weight ratio of vegetable oil-to-mineral oil from about 0 to about 2.6, and varying the weight ratio of co-surfactant-to-surfactant from about 0.89 to about 2.0, thereby forming an improved active agent-containing topical formulation.

In certain embodiments, the invention relates to any one of the aforementioned methods, wherein the ratios are varied simultaneously.

In certain embodiments, the invention relates to a method of optimizing the rate of release of an active agent from a topical formulation, comprising the step of varying the weight ratio of vegetable oil-to-mineral oil from about 0.03 to about 1.00, thereby forming an improved active agent-containing topical formulation.

In certain embodiments, the invention relates to any one of the aforementioned methods, wherein the vegetable oil contains from about 10% to about 78% polyunsaturated fatty acids.

In certain embodiments, the invention relates to any one of the aforementioned methods, wherein the rate of release of the active agent from the improved active agent-containing topical formulation is less variable over time than the rate of release of the active agent from a reference formulation.

In certain embodiments, the invention relates to any one of the aforementioned methods, wherein the active agent is a corticosteroid.

In certain embodiments, the invention relates to any one of the aforementioned methods, wherein the active agent is hydrocortisone 17-butyrate.

In certain embodiments, the invention relates to a method of treating a skin disorder, comprising the steps of:

applying topically to an area of skin of a subject in need thereof a therapeutically-effective amount of any one of the aforementioned improved active agent-containing topical formulations.

### BRIEF DESCRIPTION OF THE FIGURES

FIG. 1 tabulates various vegetable oil/mineral oil ratios used in the methods and formulations of the invention.

FIG. 2 tabulates the average % linoleic acid, the viscosity at 35° C., and the hydrophilic-lipophilic balance (HLB) of various oils used in the methods and formulations of the invention. N.A.=not applicable.

FIG. 3 tabulates the components and their weight percentages for exemplary formulations of the invention. *N.P.=not present.

FIG. 4 tabulates the effect of the ratio of vegetable oil to mineral oil on in vitro release of hydrocortisone 17-butyrate (“hydrocortisone butyrate” or “HCB”) from cream NB1231-07 (ratio=0) (see FIG. 3 and FIG. 10), and a cream vehicle with a vegetable oil/mineral oil ratio of 0.2.

FIG. 5 depicts the rate of HCB release over time for a cream vehicle with 0% vegetable oil, as compared to a cream vehicle with a vegetable oil/mineral oil ratio of 0.2. See FIG. 4.

FIG. 6 depicts the cumulative amount of HCB released over time for a cream vehicle with 0% vegetable oil, as compared to a cream vehicle with a vegetable oil/mineral oil ratio of 0.2. See FIG. 4. The x-axis is square root of time. Plotting mass/unit area versus square root of time linearizes the data. Flux (release rate) is mass/area/time this value tends to decrease with time. So, plotting cumulative release versus time generates a non linear plot.

FIG. 7 tabulates the effect of the ratio of vegetable oil to mineral oil on in vitro release of HCB from lotion R6546 (ratio=0.55) (see FIG. 3 and FIG. 10), and a lotion vehicle with a vegetable oil/mineral oil ratio of 0 (Lotion NB 1177-45 in FIG. 3 and FIG. 10).

FIG. 8 depicts the cumulative amount of HCB released over time for a lotion vehicle with 0% vegetable oil, as compared to a lotion vehicle with a vegetable oil/mineral oil ratio of 0.55. See FIG. 7. The x-axis is square root of time. Plotting mass/unit area versus square root of time linearizes the data. Flux (release rate) is mass/area/time; this value tends to decrease with time; thus, plotting cumulative release versus time generates a non-linear plot.

FIG. 9 tabulates various vegetable oil/mineral oil and co-surfactant/surfactant ratios used in the methods and formulations of the invention.

FIG. 10 tabulates the components and their weight percentages for exemplary formulations of the invention. *N.P.=not present.

FIG. 11 depicts the relationship between vegetable/mineral oil and co-surfactant/surfactant ratio on the rate of HCB release and active ingredient flux from exemplary formulations.

FIG. 12 depicts the rate of HCB release from exemplary formulations containing vegetable oils with varying concentrations of linoleic acid.

FIG. 13 depicts the predicted and actual cumulative amounts of hydrocortisone butyrate released after 4 hours for a series of exemplary formulations varying in their vegetable/mineral oil and co-surfactant/surfactant ratios.

### DETAILED DESCRIPTION OF THE INVENTION

### Overview

In certain embodiments, the invention relates to the discovery that (i) small variations in the ratio of vegetable oil-to-mineral oil in a topical formulation have a large impact on the rate of release of active agents from a formulation, and (ii) the magnitude of the impact depends upon the particular vegetable oil used. In certain embodiments, the invention relates to the discovery that systematic variation in the vegetable oil used and ratio of vegetable oil-to-mineral oil allows for tailoring of the release rate of an active agent from a topical formulation. So, in certain embodiments, the invention relates to a method for tailoring the bioavailability of an active agent in a topical formulation by varying the identity and ratio of vegetable oil-to-mineral oil in a topically applied formulation.

In certain embodiments, the invention relates to topical formulations with no limitations on the linoleic acid content of the incorporated vegetable oils; oils with a variety of linoleic acid contents are shown to be equally effective in promoting active ingredient release.

In certain embodiments, the invention relates to simultaneous systematic variation in the ratios of vegetable and mineral oils and co-surfactant/surfactant ratios to achieve a stated goal.

### DEFINITIONS

For convenience, certain terms employed in the specification and appended claims are collected here. These definitions should be read in light of the entire disclosure and understood as by a person of skill in the art.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

The phrase “or,” as used herein in the specification and in the claims, should be understood to mean “either or both” of the elements so conjoined, i.e., elements that are conjunctively present in some cases and disjunctively present in other cases. Multiple elements listed with “or” should be construed in the same fashion, i.e., “one or more” of the elements so conjoined. Other elements may optionally be present other than the elements specifically identified by the “or” clause, whether related or unrelated to those elements specifically identified. Thus, as a non-limiting example, a reference to “A or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

### Exemplary Constituents of Emulsions and Compositions of the Invention

Exemplary identities of various constituents of the compositions of the present invention are described below.

### 1. Propellants

In certain embodiments, the propellant is a HFA or a mixture of one or more hydrofluorocarbons. Suitable hydrofluorocarbons include 1,1,1,2-tetrafluoroethane (HFA 134a); 1,1,1,2,3,3,3-heptafluoropropane (HFA 227); and mixtures and admixtures of these and other HFAs that are currently approved or may become approved for medical use are suitable. The concentration of the HFA propellant is about 2% to about 50% by weight of the composition. In certain embodiments, the propellant comprises a hydrofluoroolefin (HFO), or a mixture of HFO and HFA. Suitable hydrofluoroolefins include 1,3,3,3-tetrafluoropropene (HFO 1234ze) and mixtures and admixtures of this and other HFO suitable for topical use. The concentration of the HFO propellant is about 2% to about 50% by weight of the composition. Hydrocarbon as well as CFC propellants can also be used in the present invention.

### 2. Vehicles

Suitable topical vehicles and vehicle components for use with the formulations of the invention are well known in the cosmetic and pharmaceutical arts, and include such vehicles (or vehicle components) as water; organic solvents such as alcohols (particularly lower alcohols readily capable of evaporating from the skin such as ethanol), glycols (such as propylene glycol, butylene glycol, and glycerol (glycerin)), aliphatic alcohols (such as lanolin); mixtures of water and organic solvents (such as water and alcohol), and mixtures of organic solvents such as alcohol and glycerol (optionally also with water); lipid-based materials such as fatty acids, acylglycerols (including oils, such as mineral oil, and fats of natural or synthetic origin), phosphoglycerides, sphingolipids and waxes; protein-based materials such as collagen and gelatin; silicone-based materials (both non-volatile and volatile) such as cyclomethicone, dimethiconol, dimethicone, and dimethicone copolyol; hydrocarbon-based materials such as petrolatum and squalane; and other vehicles and vehicle components that are suitable for administration to the skin, as well as mixtures of topical vehicle components as identified above or otherwise known to the art.

In one embodiment, the compositions of the present invention are oil-in-water emulsions. Liquids suitable for use in formulating compositions of the present invention include water, and water-miscible solvents such as glycols (e.g., ethylene glycol, butylene glycol, isoprene glycol, propylene glycol), glycerol, liquid polyols, dimethyl sulfoxide, and isopropyl alcohol. One or more aqueous vehicles may be present.

In one embodiment, formulations without methanol, ethanol, propanols, or butanols are desirable.

### 3. Surfactants and Emulsifiers

Many topical formulations contain chemical emulsions which use surface active ingredients (emulsifiers and surfactants) to disperse dissimilar chemicals in a particular solvent system. For example, most lipid-like (oily or fatty) or lipophilic ingredients do not uniformly disperse in aqueous solvents unless they are first combined with emulsifiers, which form microscopic aqueous soluble structures (droplets) that contain a lipophilic interior and a hydrophilic exterior, resulting in an oil-in-water emulsion. In order to be soluble in aqueous media, a molecule must be polar or charged so as to favorably interact with water molecules, which are also polar. Similarly, to dissolve an aqueous-soluble polar or charged ingredient in a largely lipid or oil-based solvent, an emulsifier is typically used which forms stable structures that contain the hydrophilic components in the interior of the structure while the exterior is lipophilic so that it can dissolve in the lipophilic solvent to form a water-in-oil emulsion. It is well known that such emulsions can be destabilized by the addition of salts or other charged ingredients which can interact with the polar or charged portions of the emulsifier within an emulsion droplet. Emulsion destabilization results in the aqueous and lipophilic ingredients separating into two layers, potentially destroying the commercial value of a topical product.

Surfactants suitable for use in the present invention may be ionic or non-ionic. These include, but are not limited to: sodium isostearate, cetyl alcohol, polysorbates (Polysorbate 20, Polysorbate 40, Polysorbate 60, Polysorbate 80), steareth-10 (Brij 76), sodium dodecyl sulfate (sodium lauryl sulfate), lauryl dimethyl amine oxide, cetyltrimethylammonium bromide (CTAB), polyethoxylated alcohols, polyoxyethylene sorbitan, octoxynol, N,N-dimethyldodecylamine-N-oxide, hexadecyltrimethylammonium bromide (HTAB), polyoxyl 10 lauryl ether, bile salts (such as sodium deoxycholate or sodium cholate), polyoxyl castor oil, nonylphenol ethoxylate, cyclodextrins, lecithin, dimethicone copolyol, lauramide DEA, cocamide DEA, cocamide MEA, oleyl betaine, cocamidopropyl betaine, cocamidopropyl phosphatidyl PG-dimonium chloride, dicetyl phosphate (dihexadecyl phosphate), ceteareth-10 phosphate, methylbenzethonium chloride, dicetyl phosphate, ceteth-10 phosphate (ceteth-10 is the polyethylene glycol ether of cetyl alcohol where n has an average value of 10; ceteth-10 phosphate is a mixture of phosphoric acid esters of ceteth-10), ceteth-20, Brij S10 (polyethylene glycol octadecyl ether, average Mₙ˜711), PEG-20 phytosterol, and Poloxamers (including, but not limited to, Poloxamer 188 (HO(C₂H₄O)ₐ(CH(CH₃)CH₂O)_{b}(C₂H₄O)ₐH, average molecular weight 8400) and Poloxamer 407 (HO(C₂H₄O)ₐ(CH(CH₃)CH₂O)_{b}(C₂H₄O)ₐH, wherein a is about 101 and b is about 56)). Appropriate combinations or mixtures of such surfactants may also be used according to the present invention.

Many of these surfactants may also serve as emulsifiers in formulations of the present invention.

Other suitable emulsifiers for use in the formulations of the present invention include, but are not limited to, glycine soja protein, sodium lauroyl lactylate, polyglyceryl-4 diisostearate-polyhydroxystearate-sebacate, behentrimonium methosulfate-cetearyl alcohol, non-ionic emulsifiers like emulsifying wax, polyoxyethylene oleyl ether, PEG-40 stearate, carbomer, cetostearyl alcohol (cetearyl alcohol), ceteareth-12, ceteareth-20, ceteareth-25, ceteareth-30, ceteareth alcohol, Ceteth-20 (Ceteth-20 is the polyethylene glycol ether of cetyl alcohol where n has an average value of 20), oleic acid, oleyl alcohol, glyceryl stearate, PEG-75 stearate, PEG-100 stearate, and PEG-100 stearate, ceramide 2, ceramide 3, stearic acid, cholesterol, laureth-12, steareth-2, and steareth-20, or combinations/mixtures thereof, as well as cationic emulsifiers like stearamidopropyl dimethylamine and behentrimonium methosulfate, or combinations/mixtures thereof.

### 4. Moisturizers, Emollients, and Humectants

One of the most important aspects of topical products in general, and cosmetic products in particular, is the consumer's perception of the aesthetic qualities of a product. For example, while white petrolatum is an excellent moisturizer and skin protectant, it is rarely used alone, especially on the face, because it is greasy, sticky, does not rub easily into the skin and may soil clothing. Consumers highly value products which are aesthetically elegant and have an acceptable tactile feel and performance on their skin.

Suitable moisturizers for use in the formulations of the present invention include, but are not limited to, lactic acid and other hydroxy acids and their salts, glycerol, propylene glycol, butylene glycol, sodium PCA, sodium hyaluronate, Carbowax 200, Carbowax 400, and Carbowax 800.

Suitable emollients or humectants for use in the formulations of the present invention include, but are not limited to, panthenol, cetyl palmitate, glycerol (glycerin), PPG-15 stearyl ether, lanolin alcohol, lanolin, lanolin derivatives, cholesterol, petrolatum, isostearyl neopentanoate, octyl stearate, mineral oil, isocetyl stearate, myristyl myristate, octyl dodecanol, 2-ethylhexyl palmitate (octyl palmitate), dimethicone, phenyl trimethicone, cyclomethicone, C₁₂-C₁₅ alkyl benzoates, dimethiconol, propylene glycol, Theobroma grandiflorum seed butter, sunflower seed oil, ceramides (e.g., ceramide 2 or ceramide 3), hydroxypropyl bispalmitamide MEA, hydroxypropyl bislauramide MEA, hydroxypropyl bisisostearamide MEA, 1,3-bis(N-2-(hydroxyethyl)stearoylamino)-2-hydroxy propane, bis-hydroxyethyl tocopherylsuccinoylamido hydroxypropane, urea, aloe, allantoin, glycyrrhetinic acid, safflower oil, oleyl alcohol, oleic acid, stearic acid, dicaprylate/dicaprate, diethyl sebacate, isostearyl alcohol, pentylene glycol, isononyl isononanoate, polyquaternium-10 (quaternized hydroxyethyl cellulose), camellia oleifera leaf extract, phytosteryl canola glycerides, shea butter, caprylic/capric triglycerides, punica granatum sterols, ethylhexyl stearate, betaine, behenyl alcohol (docosanol), stearyl alcohol (1-octadecanol), laminaria ochroleuca extract, behenic acid, caproyl sphingosine, caproyl phytosphingosine, dimethicone-divinyldimethicone-silsesquioxane crosspolymer, potassium lactate, sodium hyaluronate crosspolymer, hydrolyzed hyaluronic acid, sodium butyroyl-formoyl hyaluronate, polyglutamic acid, tetradecyl aminobutyroylvalylaminobutyric urea trifluoroacetate, micrococcus lysate, hydrolyzed rice bran protein, glycine soja protein, and 1,3-bis(N-2-(hydroxyethyl)palmitoylamino)-2-hydroxypropane.

In addition, appropriate combinations and mixtures of any of these moisturizing agents and emollients may be used in accordance with the present invention. Many of these are classified as “skin conditioners.”

### 5. Preservatives and Antioxidants

The composition may further include components adapted to improve the stability or effectiveness of the applied formulation.

Suitable preservatives for use in the present invention include, but are not limited to: ureas, such as imidazolidinyl urea and diazolidinyl urea; chlorphenesin; methylisothiazolinone; phenoxyethanol; sodium methyl paraben, methylparaben, ethylparaben, and propylparaben; ethylhexyl glycerin; potassium sorbate; sodium benzoate; sorbic acid; benzoic acid; caprylyl glycol; formaldehyde; phytosphingosine; citric acid; sodium citrate; zinc citrate; chlorine dioxide; quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; piroctone olamine; Vitis vinifera seed oil; and alcoholic agents, for example, chlorobutanol, dichlorobenzyl alcohol, phenylethyl alcohol, and benzyl alcohol.

Suitable antioxidants include, but are not limited to, ascorbic acid and its esters, sodium bisulfite, butylated hydroxytoluene, butylated hydroxyanisole, tocopherols (such as α-tocopherol), tocopheryl acetate, superoxide dismutase, oxidoreductases, Arabidopsis thaliana extract, chrysin, black raspberry seed oil, raspberry seed oil, pomegranate seed oil, cranberry seed oil, sodium ascorbate/ascorbic acid, ascorbyl palmitate, propyl gallate, and chelating agents like EDTA (e.g., disodium EDTA), citric acid, and sodium citrate.

In certain embodiments, the antioxidant or preservative comprises (3-(4-chlorophenoxy)-2-hydroxypropyl)carbamate.

In certain embodiments, antioxidants or preservatives of the present invention may also function as a moisturizer or emollient, for example.

In addition, combinations or mixtures of these preservatives or anti-oxidants may also be used in the formulations of the present invention.

### 6. Active Agents

The active agent may be any material that has a desired effect when applied topically to a mammal, particularly a human. Suitable classes of active agents include, but are not limited to, antibiotic agents, antimicrobial agents, anti-acne agents, antibacterial agents, antifungal agents, antiviral agents, steroidal anti-inflammatory agents, non-steroidal anti-inflammatory agents, anesthetic agents, antipruriginous agents, antiprotozoal agents, anti-oxidants, antihistamines, vitamins, and hormones. Mixtures of any of these active agents may also be employed. Additionally, dermatologically-acceptable salts and esters of any of these agents may be employed.

6.1 Antibiotics

Representative antibiotics include, without limitation, benzoyl peroxide, alfa terpineol, octopirox, erythromycin, zinc, tetracyclin, triclosan, azelaic acid and its derivatives, phenoxy ethanol and phenoxy propanol, ethyl acetate, clindamycin (e.g., clindamycin phosphate) and meclocycline; sebostats such as flavinoids; alpha and beta hydroxy acids; and bile salts such as scymnol sulfate and its derivatives, deoxycholate and cholate. The antibiotic can be an antifungal agent. Suitable antifungal agents include, but are not limited to, clotrimazole, econazole, ketoconazole, itraconazole, miconazole, oxiconazole, sulconazole, butenafine, naftifine, terbinafine, undecylinic acid, tolnaftate, and nystatin. Mixtures of these antibiotic agents may also be employed. Additionally, dermatologically-acceptable salts and esters of any of these agents may be employed.

6.2 Non-Steroidal Anti-Inflammatory Agents

Representative examples of non-steroidal anti-inflammatory agents include, without limitation, oxicams, such as piroxicam, isoxicam, tenoxicam, sudoxicam; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac, fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone; and niacinamide. Mixtures of these non-steroidal anti-inflammatory agents may also be employed, as well as the dermatologically acceptable salts and esters of these agents. For example, etofenamiate, a flufenamic acid derivative, is particularly useful for topical application.

6.3 Steroidal Anti-Inflammatory Agents

Representative examples of steroidal anti-inflammatory drugs include, without limitation, corticosteroids such as hydrocortisone, hydroxyl-triamcinolone, alpha-methyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, difluorosone diacetate, fluradrenolone acetonide, medrysone, amcinafel, amcinafide, betamethasone and the balance of its esters (including betamethasone dipropionate), chloroprednisone, chloroprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

6.4 Anesthetics

Suitable anesthetics include the aminoacylanilide compounds such as lidocaine, prilocaine, bupivacaine, levo-bupivacaine, ropivacaine, mepivacaine and related local anesthetic compounds having various substituents on the ring system or amine nitrogen; the aminoalkyl benzoate compounds, such as procaine, chloroprocaine, propoxycaine, hexylcaine, tetracaine, cyclomethycaine, benoxinate, butacaine, proparacaine, butamben, and related local anesthetic compounds; cocaine and related local anesthetic compounds; amino carbonate compounds such as diperodon and related local anesthetic compounds; N-phenylamidine compounds such as phenacaine and related anesthetic compounds; N-aminoalkyl amide compounds such as dibucaine and related local anesthetic compounds; aminoketone compounds such as falicaine, dyclonine and related local anesthetic compounds; and amino ether compounds such as pramoxine, dimethisoquien, and related local anesthetic compounds; and para-amino benzoic acid esters such as benzocaine. Other suitable local anesthetics include ketocaine, dibucaine, amethocaine, propanacaine, and propipocaine.

6.5 Antimicrobial Agents

Suitable antimicrobial agents include, but are not limited to, antibacterial, antifungal, antiprotozoal and antiviral agents, such as beta-lactam drugs, quinolone drugs, ciprofloxacin, norfloxacin, tetracycline, erythromycin, amikacin, triclosan, doxycycline, capreomycin, chlorhexidine, chlortetracycline, oxytetracycline, clindamycin (e.g., clindamycin phosphate), ethambutol, metronidazole, pentamidine, gentamicin, kanamycin, lineomycin, methacycline, methenamine, minocycline, neomycin, netilmicin, streptomycin, tobramycin, and miconazole. Also included are tetracycline hydrochloride, famesol, erythromycin estolate, erythromycin stearate (salt), amikacin sulfate, doxycycline hydrochloride, chlorhexidine gluconate, chlorhexidine hydrochloride, chlortetracycline hydrochloride, oxytetracycline hydrochloride, clindamycin hydrochloride, clindamycin phosphate, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, triclosan, octopirox, nystatin, tolnaftate, clotrimazole, anidulafungin, micafungin, voriconazole, lanoconazole, ciclopirox and mixtures thereof.

6.6 Keratolytic Agents

Suitable keratolytic agents include, but are not limited to, urea, salicylic acid, papain, bromelain, sulfur, glycolic acid, pyruvic acid, resorcinol, N-acetylcysteine, mandelic acid, retinoids such as retinoic acid (e.g., tretinoin) and its derivatives (e.g., cis and trans, esters), retinol, alpha hydroxy acids, beta hydroxy acids, coal tar, and combinations thereof.

### 7. Purging Gases

In one embodiment, the air in the container charged with the composition is replaced by an inert gas. In certain embodiments, the inert gas is selected from the group consisting of argon, nitrogen, and mixtures thereof.

### 8. Buffer Salts

Suitable buffer salts are well-known in the art. Examples of suitable buffer salts include, but are not limited to sodium citrate, citric acid, sodium phosphate monobasic, sodium phosphate dibasic, sodium phosphate tribasic, potassium phosphate monobasic, potassium phosphate dibasic, and potassium phosphate tribasic.

### 9. Viscosity Modifiers

Suitable viscosity adjusting agents (i.e., thickening and thinning agents or viscosity modifying agents) for use in the formulations of the present invention include, but are not limited to, protective colloids or non-ionic gums such as hydroxyethylcellulose, xanthan gum, and sclerotium gum, as well as magnesium aluminum silicate, silica, microcrystalline wax, beeswax, paraffin, and cetyl palmitate. Crosspolymers of acrylates/C₁₀₋₃₀ alkyl acrylate are also considered. In addition, appropriate combinations or mixtures of these viscosity adjusters may be utilized according to the present invention.

### 10. Additional Constituents

Additional constituents suitable for incorporation into the emulsions of the present invention include, but are not limited to: skin protectants, adsorbents, demulcents, emollients, moisturizers, sustained release materials, solubilizing agents, skin-penetration agents, skin soothing agents, deodorant agents, antiperspirants, sun screening agents, sunless tanning agents, vitamins, hair conditioning agents, anti-irritants, anti-aging agents, abrasives, absorbents, anti-caking agents, anti-static agents, astringents (e.g., witch hazel, alcohol, and herbal extracts such as chamomile extract), binders/excipients, buffering agents, chelating agents, film forming agents, conditioning agents, opacifying agents, lipids, immunomodulators, and pH adjusters (e.g., citric acid, sodium hydroxide, and sodium phosphate).

For example, lipids normally found in healthy skin (or their functional equivalents) may be incorporated into the emulsions of the present invention. In certain embodiments, the lipid is selected from the group consisting of ceramides, cholesterol, and free fatty acids. Examples of lipids include, but are not limited to, ceramide 1, ceramide 2, ceramide 3, ceramide 4, ceramide 5, ceramide 6, hydroxypropyl bispalmitamide MEA, and hydroxypropyl bislauramide MEA, and combinations thereof.

Examples of peptides that interact with protein structures of the dermal-epidermal junction include palmitoyl dipeptide-5 diaminobutyloyl hydroxythreonine, palmitoyl tripeptide-5, acetyl octapeptide-3, pentapeptide-3, palmitoyl dipeptide-5 diaminohydroxybutyrate, dipeptide diaminobutyroyl benzylamide diacetate, palmitoyl tetrapeptide-7, palmitoyl oligopeptide, and palmitoyl dipeptide-6 diaminohydroxybutyrate.

Examples of skin soothing agents include, but are not limited to algae extract, mugwort extract, stearyl glycyrrhetinate, bisabolol, allantoin, aloe, avocado oil, green tea extract, hops extract, chamomile extract, colloidal oatmeal, calamine, cucumber extract, and combinations thereof.

N-hydroxysuccinimide activates the elimination of blood originated pigments responsible for dark color and inflammation that causes under eye circles.

In certain embodiments, the compositions comprise bergamot or bergamot oil. Bergamot oil is a natural skin toner and detoxifier. In certain embodiments, it may prevent premature aging of skin and may have excellent effects on oily skin conditions and acne.

Examples of vitamins include, but are not limited to, vitamins A, D, E, K, and combinations thereof. Vitamin analogues are also contemplated; for example, the vitamin D analogues calcipotriene or calcipotriol.

In certain embodiments, the vitamin may be present as tetrahexyldecyl ascorbate. This compound exhibits anti-oxidant activity, inhibiting lipid peroxidation. In certain embodiments, use can mitigate the damaging effects of UV exposure. Studies have shown it to stimulate collagen production as well as clarifying and brightening the skin by inhibiting melanogenesis (the production of pigment) thereby promoting a more even skin tone.

Examples of sunscreens include, but are not limited to, p-aminobenzoic acid, avobenzone, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, titanium dioxide, trolamine salicylate, zinc oxide, 4-methylbenzylidene camphor, methylene bis-benzotriazolyl tetramethylbutylphenol, bis-ethylhexyloxyphenol methoxyphenyl triazine, terephthalylidene dicamphor sulfonic acid, drometrizole trisiloxane, disodium phenyl dibenzimidazole tetrasulfonate, diethylamino hydroxybenzoyl hexyl benzoate, octyl triazone, diethylhexyl butamido triazone, polysilicone-15, and combinations thereof.

Suitable fragrances and colors may be used in the formulations of the present invention. Examples of fragrances and colors suitable for use in topical products are known in the art.

Suitable immunomodulators include, but are not limited to, tetrachlorodecaoxide, deoxycholic acid, tacrolimus, pimecrolimus, and beta-glucan.

In certain embodiments, palmitoyl-lysyl-valyl-lysine bistrifluoroacetate is added. This peptide stimulates collagen synthesis in human fibroblasts.

In certain embodiments, plant extracts may be included. Examples include artemisia vulgaris extract, plankton extract, chlorella vulgaris extract, and phytosterol.

An example of a film-forming agent is polysilicone-11.

Often, one constituent of a composition may accomplish several functions. In one embodiment, the present invention relates to constituents that may act as a lubricant, an emollient, or a skin-penetrating agent. In one embodiment, the multi-functional constituent is socetyl stearate, isopropyl isostearate, isopropyl palmitate, or isopropyl myristate.

### Exemplary Formulations of the Invention

In certain embodiments, the invention relates to a formulation, wherein the formulation comprises
an active agent;
a vegetable oil; and
a mineral oil,
wherein the weight ratio of vegetable oil-to-mineral oil is about 0.03 to about 1.00.

In certain embodiments, the invention relates to a formulation, wherein the formulation comprises
an active agent; and
a mineral oil,
wherein the weight ratio of vegetable oil-to-mineral oil is from about 0 to about 2.6.

In certain embodiments, the invention relates to a formulation, wherein the formulation comprises
an active agent;
a surfactant;
a co-surfactant; and
a mineral oil,

wherein the weight ratio of co-surfactant-to-surfactant is from about 0.89 to about 2.0.

In certain embodiments, the invention relates to a formulation, wherein the formulation comprises
an active agent;
a surfactant;
a co-surfactant; and
a mineral oil,

wherein

the weight ratio of vegetable oil-to-mineral oil is from about 0 to about 2.6; and

the weight ratio of co-surfactant-to-surfactant is from about 0.89 to about 2.0.

In certain embodiments, the invention relates to any one of the aforementioned formulations, wherein the formulation is an improved active agent-containing formulation.

In certain embodiments, the invention relates to any one of the aforementioned formulations, wherein the weight ratio of vegetable oil-to-mineral oil is about 0.03, about 0.06, about 0.13, about 0.2, about 0.55, about 0.75, or about 1.00. In certain embodiments, the invention relates to any one of the aforementioned formulations, wherein the weight ratio of vegetable oil-to-mineral oil is about 0.2 or about 0.55.

In certain embodiments, the invention relates to any one of the aforementioned formulations, wherein the weight ratio of vegetable oil-to-mineral oil is about 0.0, about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, or about 2.6.

In certain embodiments, the invention relates to any one of the aforementioned formulations, wherein the weight ratio of co-surfactant-to-surfactant is about 0.89, about 1.0, about 1.1, about 1.2, about 1.3, about 1.4, about 1.5, about 1.6, about 1.7, about 1.8, about 1.9, or about 2.0.

In certain embodiments, the invention relates to any one of the aforementioned formulations, wherein the active agent is a corticosteroid.

In certain embodiments, the invention relates to any one of the aforementioned formulations, wherein the active agent is hydrocortisone 17-butyrate.

In certain embodiments, the invention relates to any one of the aforementioned formulations, wherein the vegetable oil comprises mono- and poly-unsaturated fatty acids.

In certain embodiments, the invention relates to any one of the aforementioned formulations, wherein the vegetable oil comprises mono- and poly-unsaturated fatty acids with acyl chain lengths between about 4 and about 28 carbons.

In certain embodiments, the invention relates to any one of the aforementioned formulations, wherein the vegetable oil comprises poly-unsaturated fatty acids in an amount from about 10% to about 78% of the number of fatty acids.

In certain embodiments, the invention relates to any one of the aforementioned formulations, wherein the poly-unsaturated fatty acid is linoleic acid.

In certain embodiments, the invention relates to any one of the aforementioned formulations, wherein the vegetable oil is safflower oil, sunflower oil, corn oil, sesame oil, peanut oil, canola oil, or olive oil.

In certain embodiments, the invention relates to any one of the aforementioned formulations, wherein the vegetable oil is safflower oil.

In certain embodiments, the invention relates to any one of the aforementioned formulations, wherein the vegetable oil has a viscosity between about 30 cP and about 50 cP at 35° C.

In certain embodiments, the invention relates to any one of the aforementioned formulations, wherein the vegetable oil has a HLB value from about 6 to about 8. In certain embodiments, the invention relates to any one of the aforementioned formulations, wherein the vegetable oil has a HLB value of 6, 7, or 8.

In certain embodiments, the invention relates to any one of the aforementioned formulations, wherein the mineral oil is light mineral oil.

In certain embodiments, the invention relates to any one of the aforementioned formulations, wherein the mineral oil has a viscosity from about 10 cP to about 20 cP at 35° C.

In certain embodiments, the invention relates to any one of the aforementioned formulations, wherein the mineral oil has a HLB value from about 9 to about 11. In certain embodiments, the invention relates to any one of the aforementioned formulations, wherein the mineral oil has a HLB value of 10.

In certain embodiments, the invention relates to any one of the aforementioned formulations, wherein the formulation is an oil-in-water emulsion.

In certain embodiments, the invention relates to any one of the aforementioned formulations, wherein the formulation comprises

Hydrocortisone 17-butyrate
Water
Propylparaben
Cetostearyl Alcohol
White Petrolatum
Ceteth-20
Sodium Citrate
Citric Acid
Mineral oil

In certain embodiments, the invention relates to any one of the aforementioned formulations, wherein the formulation consists essentially of

Hydrocortisone 17-butyrate
Water
Propylparaben
Cetostearyl Alcohol
White Petrolatum
Ceteth-20
Sodium Citrate
Citric Acid
Mineral oil

In certain embodiments, the invention relates to any one of the aforementioned formulations, wherein the formulation comprises, by weight of the formulation

| | |
|---|---|
| Hydrocortisone 17-butyrate | From about 0.01% to about 0.2% |
| Water | From about 15% to about 90% |
| Propylparaben | From about 0.05% to about 0.15% |
| Cetostearyl Alcohol | From about 2% to about 9% |
| White Petrolatum | From about 1.5% to about 60% |
| Ceteth-20 | From about 1% to about 9% |
| Sodium Citrate | From about 0.07% to about 0.50% |
| Citric Acid | From about 0.09% to about 0.60% |
| Mineral oil | From about 2% to about 27% |

In certain embodiments, the invention relates to any one of the aforementioned formulations, wherein the formulation consists essentially of, by weight of the formulation

| | |
|---|---|
| Hydrocortisone 17-butyrate | From about 0.01% to about 0.2% |
| Water | From about 15% to about 90% |
| Propylparaben | From about 0.05% to about 0.15% |
| Cetostearyl Alcohol | From about 2% to about 9% |
| White Petrolatum | From about 1.5% to about 60% |
| Ceteth-20 | From about 1% to about 9% |
| Sodium Citrate | From about 0.07% to about 0.50% |
| Citric Acid | From about 0.09% to about 0.60% |
| Mineral oil | From about 2% to about 27% |

In certain embodiments, the invention relates to any one of the aforementioned formulations, wherein the formulation comprises

Hydrocortisone 17-butyrate
Water
Propylparaben
Cetostearyl Alcohol
White Petrolatum
Ceteth-20
Sodium Citrate
Citric Acid

and vegetable oil and mineral oil.

In certain embodiments, the invention relates to any one of the aforementioned formulations, wherein the formulation consists essentially of

Hydrocortisone 17-butyrate
Water
Propylparaben
Cetostearyl Alcohol
White Petrolatum
Ceteth-20
Sodium Citrate
Citric Acid

and vegetable oil and mineral oil.

In certain embodiments, the invention relates to any one of the aforementioned formulations, wherein the formulation comprises, by weight of the formulation

| | |
|---|---|
| Hydrocortisone 17-butyrate | From about 0.01% to about 0.2% |
| Water | From about 15% to about 90% |
| Propylparaben | From about 0.05% to about 0.15% |
| Cetostearyl Alcohol | From about 2% to about 9% |
| White Petrolatum | From about 1.5% to about 60% |
| Ceteth-20 | From about 1% to about 9% |
| Sodium Citrate | From about 0.07% to about 0.50% |
| Citric Acid | From about 0.09% to about 0.60% |
| Vegetable oil | From about 1% to about 9% |
| Mineral oil | From about 2% to about 27% |

In certain embodiments, the invention relates to any one of the aforementioned formulations, wherein the formulation consists essentially of, by weight of the formulation

| | |
|---|---|
| Hydrocortisone 17-butyrate | From about 0.01% to about 0.2% |
| Water | From about 15% to about 90% |
| Propylparaben | From about 0.05% to about 0.15% |
| Cetostearyl Alcohol | From about 2% to about 9% |
| White Petrolatum | From about 1.5% to about 60% |
| Ceteth-20 | From about 1% to about 9% |
| Sodium Citrate | From about 0.07% to about 0.50% |
| Citric Acid | From about 0.09% to about 0.60% |
| Vegetable oil | From about 1% to about 9% |
| Mineral oil | From about 2% to about 27% |

In certain embodiments, the invention relates to any one of the aforementioned formulations, wherein the formulation comprises

Hydrocortisone 17-butyrate
Water
Glycerin
Methylparaben
Propylparaben
Cetostearyl Alcohol
Urea
Dimethicone
Vegetable oil
White Petrolatum
Mineral Oil
Ceteth-20
Butylated Hydroxytoluene
Sodium Citrate
Citric Acid

In certain embodiments, the invention relates to any one of the aforementioned formulations, wherein the formulation consists essentially of

Hydrocortisone 17-butyrate
Water
Glycerin
Methylparaben
Propylparaben
Cetostearyl Alcohol
Urea
Dimethicone
Vegetable oil
White Petrolatum
Mineral Oil
Ceteth-20
Butylated Hydroxytoluene
Sodium Citrate
Citric Acid

In certain embodiments, the invention relates to any one of the aforementioned formulations, wherein the formulation consists of

Hydrocortisone 17-butyrate
Water
Glycerin
Methylparaben
Propylparaben
Cetostearyl Alcohol
Urea
Dimethicone
Vegetable oil
White Petrolatum
Mineral Oil
Ceteth-20
Butylated Hydroxytoluene
Sodium Citrate
Citric Acid

In certain embodiments, the invention relates to any one of the aforementioned formulations, wherein the formulation comprises, by weight of the formulation

| | |
|---|---|
| Hydrocortisone 17-butyrate | From about 0.01% to about 0.2% |
| Water | From about 40% to about 70% |
| Glycerin | From about 2% to about 8% |
| Methylparaben | From about 0.1% to about 0.5% |
| Propylparaben | From about 0.05% to about 0.15% |
| Cetostearyl Alcohol | From about 2% to about 8% |
| Urea | From about 0.3% to about 0.9% |
| Dimethicone | From about 0.5% to about 1.5% |
| Vegetable oil | From about 3% to about 9% |
| White Petrolatum | From about 3% to about 9% |
| Mineral Oil | From about 6% to about 18% |
| Ceteth-20 | From about 3% to about 9% |
| Butylated Hydroxytoluene | From about 0.015% to about 0.045% |
| Sodium Citrate | From about 0.015% to about 0.045% |
| Citric Acid | From about 0.2% to about 0.6% |

In certain embodiments, the invention relates to any one of the aforementioned formulations, wherein the formulation consists essentially of, by weight of the formulation

| | |
|---|---|
| Hydrocortisone 17-butyrate | From about 0.01% to about 0.2% |
| Water | From about 40% to about 70% |
| Glycerin | From about 2% to about 8% |
| Methylparaben | From about 0.1% to about 0.5% |
| Propylparaben | From about 0.05% to about 0.15% |
| Cetostearyl Alcohol | From about 2% to about 8% |
| Urea | From about 0.3% to about 0.9% |
| Dimethicone | From about 0.5% to about 1.5% |
| Vegetable oil | From about 3% to about 9% |
| White Petrolatum | From about 3% to about 9% |
| Mineral Oil | From about 6% to about 18% |
| Ceteth-20 | From about 3% to about 9% |
| Butylated Hydroxytoluene | From about 0.015% to about 0.045% |
| Sodium Citrate | From about 0.015% to about 0.045% |
| Citric Acid | From about 0.2% to about 0.6% |

In certain embodiments, the invention relates to any one of the aforementioned formulations, wherein the formulation consists of, by weight of the formulation

| | |
|---|---|
| Hydrocortisone 17-butyrate | From about 0.01% to about 0.2% |
| Water | From about 40% to about 70% |
| Glycerin | From about 2% to about 8% |
| Methylparaben | From about 0.1% to about 0.5% |
| Propylparaben | From about 0.05% to about 0.15% |
| Cetostearyl Alcohol | From about 2% to about 8% |
| Urea | From about 0.3% to about 0.9% |
| Dimethicone | From about 0.5% to about 1.5% |
| Vegetable oil | From about 3% to about 9% |
| White Petrolatum | From about 3% to about 9% |
| Mineral Oil | From about 6% to about 18% |
| Ceteth-20 | From about 3% to about 9% |
| Butylated Hydroxytoluene | From about 0.015% to about 0.045% |
| Sodium Citrate | From about 0.015% to about 0.045% |
| Citric Acid | From about 0.2% to about 0.6% |

In certain embodiments, the invention relates to any one of the aforementioned formulations, wherein the formulation comprises

| | |
|---|---|
| Hydrocortisone 17-butyrate | About 0.1% |
| Water | About 56.45% |
| Glycerin | About 5.00% |
| Methylparaben | About 0.30% |
| Propylparaben | About 0.10% |
| Cetostearyl Alcohol | About 5.34% |
| Urea | About 0.64% |
| Dimethicone | About 0.92% |
| Vegetable oil | About 6.18% |
| White Petrolatum | About 6.87% |
| Mineral Oil | About 11.33% |
| Ceteth-20 | About 6.00% |
| Butylated Hydroxytoluene | About 0.03% |
| Sodium Citrate | About 0.32% |
| Citric Acid | About 0.42% |

In certain embodiments, the invention relates to any one of the aforementioned formulations, wherein the formulation consists essentially of

| | |
|---|---|
| Hydrocortisone 17-butyrate | About 0.1% |
| Water | About 56.45% |
| Glycerin | About 5.00% |
| Methylparaben | About 0.30% |
| Propylparaben | About 0.10% |
| Cetostearyl Alcohol | About 5.34% |
| Urea | About 0.64% |
| Dimethicone | About 0.92% |
| Vegetable oil | About 6.18% |
| White Petrolatum | About 6.87% |
| Mineral Oil | About 11.33% |
| Ceteth-20 | About 6.00% |
| Butylated Hydroxytoluene | About 0.03% |
| Sodium Citrate | About 0.32% |
| Citric Acid | About 0.42% |

In certain embodiments, the invention relates to any one of the aforementioned formulations, wherein the formulation consists of

| | |
|---|---|
| Hydrocortisone 17-butyrate | About 0.1% |
| Water | About 56.45% |
| Glycerin | About 5.00% |
| Methylparaben | About 0.30% |
| Propylparaben | About 0.10% |
| Cetostearyl Alcohol | About 5.34% |
| Urea | About 0.64% |
| Dimethicone | About 0.92% |
| Vegetable oil | About 6.18% |
| White Petrolatum | About 6.87% |
| Mineral Oil | About 11.33% |
| Ceteth-20 | About 6.00% |
| Butylated Hydroxytoluene | About 0.03% |
| Sodium Citrate | About 0.32% |
| Citric Acid | About 0.42% |

In certain embodiments, the invention relates to any one of the aforementioned formulations, wherein the formulation comprises

Hydrocortisone 17-butyrate
Water
Butylparaben
Propylparaben
Cetostearyl Alcohol
Vegetable Oil
White Petrolatum
Mineral Oil
Ceteth-20
Sodium Citrate
Citric Acid

In certain embodiments, the invention relates to any one of the aforementioned formulations, wherein the formulation consists essentially of

Hydrocortisone 17-butyrate
Water
Butylparaben
Propylparaben
Cetostearyl Alcohol
Vegetable Oil
White Petrolatum
Mineral Oil
Ceteth-20
Sodium Citrate
Citric Acid

In certain embodiments, the invention relates to any one of the aforementioned formulations, wherein the formulation consists of

Hydrocortisone 17-butyrate
Water
Butylparaben
Propylparaben
Cetostearyl Alcohol
Vegetable Oil
White Petrolatum
Mineral Oil
Ceteth-20
Sodium Citrate
Citric Acid

In certain embodiments, the invention relates to any one of the aforementioned formulations, wherein the formulation comprises, by weight of the formulation,

| | |
|---|---|
| Hydrocortisone 17-butyrate | From about 0.01% to about 0.2% |
| Water | From about 15% to about 45% |
| Butylparaben | From about 0.02% to about 0.08% |
| Propylparaben | From about 0.05% to about 0.15% |
| Cetostearyl Alcohol | From about 3% to about 9% |
| Vegetable Oil | From about 1.5% to about 4.5% |
| White Petrolatum | From about 20% to about 60% |
| Mineral Oil | From about 8% to about 22% |
| Ceteth-20 | From about 1.5% to about 4.5% |
| Sodium Citrate | From about 0.06% to about 0.22% |
| Citric Acid | From about 0.09% to about 0.27% |

In certain embodiments, the invention relates to any one of the aforementioned formulations, wherein the formulation consists essentially of, by weight of the formulation,

| | |
|---|---|
| Hydrocortisone 17-butyrate | From about 0.01% to about 0.2% |
| Water | From about 15% to about 45% |
| Butylparaben | From about 0.02% to about 0.08% |
| Propylparaben | From about 0.05% to about 0.15% |
| Cetostearyl Alcohol | From about 3% to about 9% |
| Vegetable Oil | From about 1.5% to about 4.5% |
| White Petrolatum | From about 20% to about 60% |
| Mineral Oil | From about 8% to about 22% |
| Ceteth-20 | From about 1.5% to about 4.5% |
| Sodium Citrate | From about 0.06% to about 0.22% |
| Citric Acid | From about 0.09% to about 0.27% |

In certain embodiments, the invention relates to any one of the aforementioned formulations, wherein the formulation consists of, by weight of the formulation,

| | |
|---|---|
| Hydrocortisone 17-butyrate | From about 0.01% to about 0.2% |
| Water | From about 15% to about 45% |
| Butylparaben | From about 0.02% to about 0.08% |
| Propylparaben | From about 0.05% to about 0.15% |
| Cetostearyl Alcohol | From about 3% to about 9% |
| Vegetable Oil | From about 1.5% to about 4.5% |
| White Petrolatum | From about 20% to about 60% |
| Mineral Oil | From about 8% to about 22% |
| Ceteth-20 | From about 1.5% to about 4.5% |
| Sodium Citrate | From about 0.06% to about 0.22% |
| Citric Acid | From about 0.09% to about 0.27% |

In certain embodiments, the invention relates to any one of the aforementioned formulations, wherein the formulation comprises, by weight of the formulation,

| | |
|---|---|
| Hydrocortisone 17-butyrate | About 0.1% |
| Water | About 30.43% |
| Butylparaben | About 0.05% |
| Propylparaben | About 0.10% |
| Cetostearyl Alcohol | About 6.00% |
| Vegetable Oil | About 3.00% |
| White Petrolatum | About 42.00% |
| Mineral Oil | About 15.00% |
| Ceteth-20 | About 3.00% |
| Sodium Citrate | About 0.14% |
| Citric Acid | About 0.18% |

In certain embodiments, the invention relates to any one of the aforementioned formulations, wherein the formulation consists essentially of, by weight of the formulation,

| | |
|---|---|
| Hydrocortisone 17-butyrate | About 0.1% |
| Water | About 30.43% |
| Butylparaben | About 0.05% |
| Propylparaben | About 0.10% |
| Cetostearyl Alcohol | About 6.00% |
| Vegetable Oil | About 3.00% |
| White Petrolatum | About 42.00% |
| Mineral Oil | About 15.00% |
| Ceteth-20 | About 3.00% |
| Sodium Citrate | About 0.14% |
| Citric Acid | About 0.18% |

In certain embodiments, the invention relates to any one of the aforementioned formulations, wherein the formulation consists of, by weight of the formulation,

| | |
|---|---|
| Hydrocortisone 17-butyrate | About 0.1% |
| Water | About 30.43% |
| Butylparaben | About 0.05% |
| Propylparaben | About 0.10% |
| Cetostearyl Alcohol | About 6.00% |
| Vegetable Oil | About 3.00% |
| White Petrolatum | About 42.00% |
| Mineral Oil | About 15.00% |
| Ceteth-20 | About 3.00% |
| Sodium Citrate | About 0.14% |
| Citric Acid | About 0.18% |

In certain embodiments, the invention relates to any one of the aforementioned formulations, wherein the formulation comprises

Hydrocortisone 17-butyrate
Water
Butylparaben
Propylparaben
Cetostearyl Alcohol
vegetable Oil
White Petrolatum
Mineral Oil
Ceteth-20
Butylated Hydroxytoluene
Sodium Citrate
Citric Acid

In certain embodiments, the invention relates to any one of the aforementioned formulations, wherein the formulation consists essentially of

Hydrocortisone 17-butyrate
Water
Butylparaben
Propylparaben
Cetostearyl Alcohol
vegetable Oil
White Petrolatum
Mineral Oil
Ceteth-20
Butylated Hydroxytoluene
Sodium Citrate
Citric Acid

In certain embodiments, the invention relates to any one of the aforementioned formulations, wherein the formulation consists of

Hydrocortisone 17-butyrate
Water
Butylparaben
Propylparaben
Cetostearyl Alcohol
vegetable Oil
White Petrolatum
Mineral Oil
Ceteth-20
Butylated Hydroxytoluene
Sodium Citrate
Citric Acid

In certain embodiments, the invention relates to any one of the aforementioned formulations, wherein the formulation comprises, by weight of the formulation

| | |
|---|---|
| Hydrocortisone 17-butyrate | From about 0.01% to about 0.2% |
| Water | From about 45% to about 95% |
| Butylparaben | From about 0.02% to about 0.08% |
| Propylparaben | From about 0.05% to about 0.15% |
| Cetostearyl Alcohol | From about 2% to about 6% |
| vegetable Oil | From about 1.5% to about 4.5% |
| White Petrolatum | From about 1% to about 5% |
| Mineral Oil | From about 2% to about 8% |
| Ceteth-20 | From about 1% to about 3% |
| Butylated Hydroxytoluene | From about 0.01% to about 0.03% |
| Sodium Citrate | From about 0.15% to about 0.45% |
| Citric Acid | From about 0.2% to about 0.6% |

In certain embodiments, the invention relates to any one of the aforementioned formulations, wherein the formulation consists essentially of, by weight of the formulation

| | |
|---|---|
| Hydrocortisone 17-butyrate | From about 0.01% to about 0.2% |
| Water | From about 45% to about 95% |
| Butylparaben | From about 0.02% to about 0.08% |
| Propylparaben | From about 0.05% to about 0.15% |
| Cetostearyl Alcohol | From about 2% to about 6% |
| vegetable Oil | From about 1.5% to about 4.5% |
| White Petrolatum | From about 1% to about 5% |
| Mineral Oil | From about 2% to about 8% |
| Ceteth-20 | From about 1% to about 3% |
| Butylated Hydroxytoluene | From about 0.01% to about 0.03% |
| Sodium Citrate | From about 0.15% to about 0.45% |
| Citric Acid | From about 0.2% to about 0.6% |

In certain embodiments, the invention relates to any one of the aforementioned formulations, wherein the formulation consists of, by weight of the formulation

| | |
|---|---|
| Hydrocortisone 17-butyrate | From about 0.01% to about 0.2% |
| Water | From about 45% to about 95% |
| Butylparaben | From about 0.02% to about 0.08% |
| Propylparaben | From about 0.05% to about 0.15% |
| Cetostearyl Alcohol | From about 2% to about 6% |
| vegetable Oil | From about 1.5% to about 4.5% |
| White Petrolatum | From about 1% to about 5% |
| Mineral Oil | From about 2% to about 8% |
| Ceteth-20 | From about 1% to about 3% |
| Butylated Hydroxytoluene | From about 0.01% to about 0.03% |
| Sodium Citrate | From about 0.15% to about 0.45% |
| Citric Acid | From about 0.2% to about 0.6% |

In certain embodiments, the invention relates to any one of the aforementioned formulations, wherein the formulation comprises, by weight of the formulation,

| | |
|---|---|
| Hydrocortisone 17-butyrate | About 0.1% |
| Water | About 81.99% |
| Butylparaben | About 0.05% |
| Propylparaben | About 0.10% |
| Cetostearyl Alcohol | About 4.00% |
| Vegetable Oil | About 3.00% |
| White Petrolatum | About 2.50% |
| Mineral Oil | About 5.50% |
| Ceteth-20 | About 2.00% |
| Butylated Hydroxytoluene | About 0.02% |
| Sodium Citrate | About 0.32% |
| Citric Acid | About 0.42% |

In certain embodiments, the invention relates to any one of the aforementioned formulations, wherein the formulation consists essentially of, by weight of the formulation,

| | |
|---|---|
| Hydrocortisone 17-butyrate | About 0.1% |
| Water | About 81.99% |
| Butylparaben | About 0.05% |
| Propylparaben | About 0.10% |
| Cetostearyl Alcohol | About 4.00% |
| Vegetable Oil | About 3.00% |
| White Petrolatum | About 2.50% |
| Mineral Oil | About 5.50% |
| Ceteth-20 | About 2.00% |
| Butylated Hydroxytoluene | About 0.02% |
| Sodium Citrate | About 0.32% |
| Citric Acid | About 0.42% |

In certain embodiments, the invention relates to any one of the aforementioned formulations, wherein the formulation consists of, by weight of the formulation,

| | |
|---|---|
| Hydrocortisone 17-butyrate | About 0.1% |
| Water | About 81.99% |
| Butylparaben | About 0.05% |
| Propylparaben | About 0.10% |
| Cetostearyl Alcohol | About 4.00% |
| Vegetable Oil | About 3.00% |
| White Petrolatum | About 2.50% |
| Mineral Oil | About 5.50% |
| Ceteth-20 | About 2.00% |
| Butylated Hydroxytoluene | About 0.02% |
| Sodium Citrate | About 0.32% |
| Citric Acid | About 0.42% |

In certain embodiments, the invention relates to any one of the aforementioned formulations, wherein the formulation comprises

Hydrocortisone 17-butyrate
Water
Butylparaben
Propylparaben
Cetostearyl Alcohol
Vegetable oil
White Petrolatum
Mineral Oil
Ceteth-20
Sodium Citrate
Citric Acid

In certain embodiments, the invention relates to any one of the aforementioned formulations, wherein the formulation consists essentially of

Hydrocortisone 17-butyrate
Water
Butylparaben
Propylparaben
Cetostearyl Alcohol
Vegetable oil
White Petrolatum
Mineral Oil
Ceteth-20
Sodium Citrate
Citric Acid

In certain embodiments, the invention relates to any one of the aforementioned formulations, wherein the formulation consists of

Hydrocortisone 17-butyrate
Water
Butylparaben
Propylparaben
Cetostearyl Alcohol
Vegetable oil
White Petrolatum
Mineral Oil
Ceteth-20
Sodium Citrate
Citric Acid

In certain embodiments, the invention relates to any one of the aforementioned formulations, wherein the formulation comprises, by weight of the formulation

| | |
|---|---|
| Hydrocortisone 17-butyrate | From about 0.01% to about 0.2% |
| Water | From about 15% to about 45% |
| Butylparaben | From about 0.02% to about 0.08% |
| Propylparaben | From about 0.05% to about 0.15% |
| Cetostearyl Alcohol | From about 3% to about 9% |
| Vegetable oil | From about 1.5% to about 4.5% |
| White Petrolatum | From about 20% to about 60% |
| Mineral Oil | From about 7% to about 22% |
| Ceteth-20 | From about 1.5% to about 4.5% |
| Sodium Citrate | From about 0.05% to about 0.2% |
| Citric Acid | From about 0.05% to about 0.3% |

In certain embodiments, the invention relates to any one of the aforementioned formulations, wherein the formulation consists essentially of, by weight of the formulation

| | |
|---|---|
| Hydrocortisone 17-butyrate | From about 0.01% to about 0.2% |
| Water | From about 15% to about 45% |
| Butylparaben | From about 0.02% to about 0.08% |
| Propylparaben | From about 0.05% to about 0.15% |
| Cetostearyl Alcohol | From about 3% to about 9% |
| Vegetable oil | From about 1.5% to about 4.5% |
| White Petrolatum | From about 20% to about 60% |
| Mineral Oil | From about 7% to about 22% |
| Ceteth-20 | From about 1.5% to about 4.5% |
| Sodium Citrate | From about 0.05% to about 0.2% |
| Citric Acid | From about 0.05% to about 0.3% |

In certain embodiments, the invention relates to any one of the aforementioned formulations, wherein the formulation consists of, by weight of the formulation

| | |
|---|---|
| Hydrocortisone 17-butyrate | From about 0.01% to about 0.2% |
| Water | From about 15% to about 45% |
| Butylparaben | From about 0.02% to about 0.08% |
| Propylparaben | From about 0.05% to about 0.15% |
| Cetostearyl Alcohol | From about 3% to about 9% |
| Vegetable oil | From about 1.5% to about 4.5% |
| White Petrolatum | From about 20% to about 60% |
| Mineral Oil | From about 7% to about 22% |
| Ceteth-20 | From about 1.5% to about 4.5% |
| Sodium Citrate | From about 0.05% to about 0.2% |
| Citric Acid | From about 0.05% to about 0.3% |

In certain embodiments, the invention relates to any one of the aforementioned formulations, wherein the formulation comprises

| | |
|---|---|
| Hydrocortisone 17-butyrate | About 0.1% |
| Water | About 30% |
| Butylparaben | About 0.05% |
| Propylparaben | About 0.1% |
| Cetostearyl Alcohol | About 6.0% |
| Vegetable oil | About 3.0% |
| White Petrolatum | About 42% |
| Mineral Oil | About 15% |
| Ceteth-20 | About 3.0% |
| Sodium Citrate | About 0.14% |
| Citric Acid | About 0.18% |

In certain embodiments, the invention relates to any one of the aforementioned formulations, wherein the formulation consists essentially of

| | |
|---|---|
| Hydrocortisone 17-butyrate | About 0.1% |
| Water | About 30% |
| Butylparaben | About 0.05% |
| Propylparaben | About 0.1% |
| Cetostearyl Alcohol | About 6.0% |
| Vegetable oil | About 3.0% |
| White Petrolatum | About 42% |
| Mineral Oil | About 15% |
| Ceteth-20 | About 3.0% |
| Sodium Citrate | About 0.14% |
| Citric Acid | About 0.18% |

In certain embodiments, the invention relates to any one of the aforementioned formulations, wherein the formulation consists of

| | |
|---|---|
| Hydrocortisone 17-butyrate | About 0.1% |
| Water | About 30% |
| Butylparaben | About 0.05% |
| Propylparaben | About 0.1% |
| Cetostearyl Alcohol | About 6.0% |
| Vegetable oil | About 3.0% |
| White Petrolatum | About 42% |
| Mineral Oil | About 15% |
| Ceteth-20 | About 3.0% |
| Sodium Citrate | About 0.14% |
| Citric Acid | About 0.18% |

In certain embodiments, the invention relates to any one of the aforementioned formulations, wherein the formulation comprises

Hydrocortisone 17-butyrate
Water
Butylparaben
Propylparaben
Cetostearyl Alcohol
Vegetable oil
White Petrolatum
Mineral Oil
Ceteth-20
Butylated hydroxytoluene
Sodium Citrate
Citric Acid

In certain embodiments, the invention relates to any one of the aforementioned formulations, wherein the formulation consists essentially of

Hydrocortisone 17-butyrate
Water
Butylparaben
Propylparaben
Cetostearyl Alcohol
Vegetable oil
White Petrolatum
Mineral Oil
Ceteth-20
Butylated hydroxytoluene
Sodium Citrate
Citric Acid

In certain embodiments, the invention relates to any one of the aforementioned formulations, wherein the formulation consists of

Hydrocortisone 17-butyrate
Water
Butylparaben
Propylparaben
Cetostearyl Alcohol
Vegetable oil
White Petrolatum
Mineral Oil
Ceteth-20
Butylated hydroxytoluene
Sodium Citrate
Citric Acid

In certain embodiments, the invention relates to any one of the aforementioned formulations, wherein the formulation comprises, by weight of the formulation

| | |
|---|---|
| Hydrocortisone 17-butyrate | From about 0.01% to about 0.2% |
| Water | From about 42% to about 90% |
| Butylparaben | From about 0.02% to about 0.08% |
| Propylparaben | From about 0.05% to about 0.15% |
| Cetostearyl Alcohol | From about 2% to about 6% |
| Vegetable oil | From about 1.5% to about 4.5% |
| White Petrolatum | From about 1.0% to about 4.0% |
| Mineral Oil | From about 3.0% to about 8.0% |
| Ceteth-20 | From about 1.0% to about 3.0% |
| Butylated hydroxytoluene | From about 0.01% to about 0.03% |
| Sodium Citrate | From about 0.15% to about 0.45% |
| Citric Acid | From about 0.2% to about 0.6% |

In certain embodiments, the invention relates to any one of the aforementioned formulations, wherein the formulation consists essentially of, by weight of the formulation

| | |
|---|---|
| Hydrocortisone 17-butyrate | From about 0.01% to about 0.2% |
| Water | From about 42% to about 90% |
| Butylparaben | From about 0.02% to about 0.08% |
| Propylparaben | From about 0.05% to about 0.15% |
| Cetostearyl Alcohol | From about 2% to about 6% |
| Vegetable oil | From about 1.5% to about 4.5% |
| White Petrolatum | From about 1.0% to about 4.0% |
| Mineral Oil | From about 3.0% to about 8.0% |
| Ceteth-20 | From about 1.0% to about 3.0% |
| Butylated hydroxytoluene | From about 0.01% to about 0.03% |
| Sodium Citrate | From about 0.15% to about 0.45% |
| Citric Acid | From about 0.2% to about 0.6% |

In certain embodiments, the invention relates to any one of the aforementioned formulations, wherein the formulation consists of, by weight of the formulation

| | |
|---|---|
| Hydrocortisone 17-butyrate | From about 0.01% to about 0.2% |
| Water | From about 42% to about 90% |
| Butylparaben | From about 0.02% to about 0.08% |
| Propylparaben | From about 0.05% to about 0.15% |
| Cetostearyl Alcohol | From about 2% to about 6% |
| Vegetable oil | From about 1.5% to about 4.5% |
| White Petrolatum | From about 1.0% to about 4.0% |
| Mineral Oil | From about 3.0% to about 8.0% |
| Ceteth-20 | From about 1.0% to about 3.0% |
| Butylated hydroxytoluene | From about 0.01% to about 0.03% |
| Sodium Citrate | From about 0.15% to about 0.45% |
| Citric Acid | From about 0.2% to about 0.6% |

In certain embodiments, the invention relates to any one of the aforementioned formulations, wherein the formulation comprises

| | |
|---|---|
| Hydrocortisone 17-butyrate | About 0.1% |
| Water | About 82% |
| Butylparaben | About 0.05% |
| Propylparaben | About 0.1% |
| Cetostearyl Alcohol | About 4.0% |
| Vegetable oil | About 3.0% |
| White Petrolatum | About 2.5% |
| Mineral Oil | About 5.5% |
| Ceteth-20 | About 2.0% |
| Butylated hydroxytoluene | About 0.02% |
| Sodium Citrate | About 0.32% |
| Citric Acid | About 0.42% |

In certain embodiments, the invention relates to any one of the aforementioned formulations, wherein the formulation consists essentially of

| | |
|---|---|
| Hydrocortisone 17-butyrate | About 0.1% |
| Water | About 82% |
| Butylparaben | About 0.05% |
| Propylparaben | About 0.1% |
| Cetostearyl Alcohol | About 4.0% |
| Vegetable oil | About 3.0% |
| White Petrolatum | About 2.5% |
| Mineral Oil | About 5.5% |
| Ceteth-20 | About 2.0% |
| Butylated hydroxytoluene | About 0.02% |
| Sodium Citrate | About 0.32% |
| Citric Acid | About 0.42% |

In certain embodiments, the invention relates to any one of the aforementioned formulations, wherein the formulation consists of

| | |
|---|---|
| Hydrocortisone 17-butyrate | About 0.1% |
| Water | About 82% |
| Butylparaben | About 0.05% |
| Propylparaben | About 0.1% |
| Cetostearyl Alcohol | About 4.0% |
| Vegetable oil | About 3.0% |
| White Petrolatum | About 2.5% |
| Mineral Oil | About 5.5% |
| Ceteth-20 | About 2.0% |
| Butylated hydroxytoluene | About 0.02% |
| Sodium Citrate | About 0.32% |
| Citric Acid | About 0.42% |

In certain embodiments, the invention relates to any one of the aforementioned formulations, wherein the formulation comprises

Hydrocortisone 17-butyrate
Water
Glycerin
Methylparaben
Propylparaben
Cetostearyl Alcohol
Urea
Dimethicone
White Petrolatum
Mineral Oil
Ceteth-20
Butylated hydroxytoluene
Sodium Citrate
Citric Acid

In certain embodiments, the invention relates to any one of the aforementioned formulations, wherein the formulation consists essentially of

Hydrocortisone 17-butyrate
Water
Glycerin
Methylparaben
Propylparaben
Cetostearyl Alcohol
Urea
Dimethicone
White Petrolatum
Mineral Oil
Ceteth-20
Butylated hydroxytoluene
Sodium Citrate
Citric Acid

In certain embodiments, the invention relates to any one of the aforementioned formulations, wherein the formulation consists of

Hydrocortisone 17-butyrate
Water
Glycerin
Methylparaben
Propylparaben
Cetostearyl Alcohol
Urea
Dimethicone
White Petrolatum
Mineral Oil
Ceteth-20
Butylated hydroxytoluene
Sodium Citrate
Citric Acid

In certain embodiments, the invention relates to any one of the aforementioned formulations, wherein the formulation comprises, by weight of the formulation

| | |
|---|---|
| Hydrocortisone 17-butyrate | From about 0.01% to about 0.2% |
| Water | From about 30% to about 86% |
| Glycerin | From about 2.5% to about 7.5% |
| Methylparaben | From about 0.15% to about 0.45% |
| Propylparaben | From about 0.05% to about 0.15% |
| Cetostearyl Alcohol | From about 3.0% to about 11.0% |
| Urea | From about 0.3% to about 0.9% |
| Dimethicone | From about 0.5% to about 1.5% |
| White Petrolatum | From about 3.0% to about 11.0% |
| Mineral Oil | From about 9.0% to about 27.0% |
| Ceteth-20 | From about 2.0% to about 6.0% |
| Butylated hydroxytoluene | From about 0.01% to about 0.05% |
| Sodium Citrate | From about 0.15% to about 0.45% |
| Citric Acid | From about 0.2% to about 0.6% |

In certain embodiments, the invention relates to any one of the aforementioned formulations, wherein the formulation consists essentially of, by weight of the formulation

| | |
|---|---|
| Hydrocortisone 17-butyrate | From about 0.01% to about 0.2% |
| Water | From about 30% to about 86% |
| Glycerin | From about 2.5% to about 7.5% |
| Methylparaben | From about 0.15% to about 0.45% |
| Propylparaben | From about 0.05% to about 0.15% |
| Cetostearyl Alcohol | From about 3.0% to about 11.0% |
| Urea | From about 0.3% to about 0.9% |
| Dimethicone | From about 0.5% to about 1.5% |
| White Petrolatum | From about 3.0% to about 11.0% |
| Mineral Oil | From about 9.0% to about 27.0% |
| Ceteth-20 | From about 2.0% to about 6.0% |
| Butylated hydroxytoluene | From about 0.01% to about 0.05% |
| Sodium Citrate | From about 0.15% to about 0.45% |
| Citric Acid | From about 0.2% to about 0.6% |

In certain embodiments, the invention relates to any one of the aforementioned formulations, wherein the formulation consists of, by weight of the formulation

| | |
|---|---|
| Hydrocortisone 17-butyrate | From about 0.01% to about 0.2% |
| Water | From about 30% to about 86% |
| Glycerin | From about 2.5% to about 7.5% |
| Methylparaben | From about 0.15% to about 0.45% |
| Propylparaben | From about 0.05% to about 0.15% |
| Cetostearyl Alcohol | From about 3.0% to about 11.0% |
| Urea | From about 0.3% to about 0.9% |
| Dimethicone | From about 0.5% to about 1.5% |
| White Petrolatum | From about 3.0% to about 11.0% |
| Mineral Oil | From about 9.0% to about 27.0% |
| Ceteth-20 | From about 2.0% to about 6.0% |
| Butylated hydroxytoluene | From about 0.01% to about 0.05% |
| Sodium Citrate | From about 0.15% to about 0.45% |
| Citric Acid | From about 0.2% to about 0.6% |

In certain embodiments, the invention relates to any one of the aforementioned formulations, wherein the formulation comprises

| | |
|---|---|
| Hydrocortisone 17-butyrate | About 0.1% |
| Water | About 54% |
| Glycerine | About 5.0% |
| Methylparaben | About 0.3% |
| Propylparaben | About 0.1% |
| Cetostearyl Alcohol | About 7.6% |
| Urea | About 0.64% |
| Dimethicone | About 0.92% |
| White Petrolatum | About 6.9% |
| Mineral Oil | About 17.5% |
| Ceteth-20 | About 3.8% |
| Butylated hydroxytoluene | About 0.03% |
| Sodium Citrate | About 0.32% |
| Citric Acid | About 0.42% |

In certain embodiments, the invention relates to any one of the aforementioned formulations, wherein the formulation consists essentially of

| | |
|---|---|
| Hydrocortisone 17-butyrate | About 0.1% |
| Water | About 54% |
| Glycerine | About 5.0% |
| Methylparaben | About 0.3% |
| Propylparaben | About 0.1% |
| Cetostearyl Alcohol | About 7.6% |
| Urea | About 0.64% |
| Dimethicone | About 0.92% |
| White Petrolatum | About 6.9% |
| Mineral Oil | About 17.5% |
| Ceteth-20 | About 3.8% |
| Butylated hydroxytoluene | About 0.03% |
| Sodium Citrate | About 0.32% |
| Citric Acid | About 0.42% |

In certain embodiments, the invention relates to any one of the aforementioned formulations, wherein the formulation consists of

| | |
|---|---|
| Hydrocortisone 17-butyrate | About 0.1% |
| Water | About 54% |
| Glycerine | About 5.0% |
| Methylparaben | About 0.3% |
| Propylparaben | About 0.1% |
| Cetostearyl Alcohol | About 7.6% |
| Urea | About 0.64% |
| Dimethicone | About 0.92% |
| White Petrolatum | About 6.9% |
| Mineral Oil | About 17.5% |
| Ceteth-20 | About 3.8% |
| Butylated hydroxytoluene | About 0.03% |
| Sodium Citrate | About 0.32% |
| Citric Acid | About 0.42% |

In certain embodiments, the invention relates to any one of the aforementioned formulations, wherein the formulation comprises, by weight of the formulation

| | |
|---|---|
| Hydrocortisone 17-butyrate | From about 0.01% to about 0.2% |
| Water | From about 30% to about 86% |
| Glycerin | From about 2.5% to about 7.5% |
| Methylparaben | From about 0.15% to about 0.45% |
| Propylparaben | From about 0.05% to about 0.15% |
| Cetostearyl Alcohol | From about 2.0% to about 8.0% |
| Urea | From about 0.3% to about 0.9% |
| Dimethicone | From about 0.5% to about 1.5% |
| White Petrolatum | From about 3.0% to about 11.0% |
| Mineral Oil | From about 9.0% to about 27.0% |
| Ceteth-20 | From about 3.0% to about 9.0% |
| Butylated hydroxytoluene | From about 0.01% to about 0.05% |
| Sodium Citrate | From about 0.15% to about 0.45% |
| Citric Acid | From about 0.2% to about 0.6% |

In certain embodiments, the invention relates to any one of the aforementioned formulations, wherein the formulation consists essentially of, by weight of the formulation

| | |
|---|---|
| Hydrocortisone 17-butyrate | From about 0.01% to about 0.2% |
| Water | From about 30% to about 86% |
| Glycerin | From about 2.5% to about 7.5% |
| Methylparaben | From about 0.15% to about 0.45% |
| Propylparaben | From about 0.05% to about 0.15% |
| Cetostearyl Alcohol | From about 2.0% to about 8.0% |
| Urea | From about 0.3% to about 0.9% |
| Dimethicone | From about 0.5% to about 1.5% |
| White Petrolatum | From about 3.0% to about 11.0% |
| Mineral Oil | From about 9.0% to about 27.0% |
| Ceteth-20 | From about 3.0% to about 9.0% |
| Butylated hydroxytoluene | From about 0.01% to about 0.05% |
| Sodium Citrate | From about 0.15% to about 0.45% |
| Citric Acid | From about 0.2% to about 0.6% |

In certain embodiments, the invention relates to any one of the aforementioned formulations, wherein the formulation consists of, by weight of the formulation

| | |
|---|---|
| Hydrocortisone 17-butyrate | From about 0.01% to about 0.2% |
| Water | From about 30% to about 86% |
| Glycerin | From about 2.5% to about 7.5% |
| Methylparaben | From about 0.15% to about 0.45% |
| Propylparaben | From about 0.05% to about 0.15% |
| Cetostearyl Alcohol | From about 2.0% to about 8.0% |
| Urea | From about 0.3% to about 0.9% |
| Dimethicone | From about 0.5% to about 1.5% |
| White Petrolatum | From about 3.0% to about 11.0% |
| Mineral Oil | From about 9.0% to about 27.0% |
| Ceteth-20 | From about 3.0% to about 9.0% |
| Butylated hydroxytoluene | From about 0.01% to about 0.05% |
| Sodium Citrate | From about 0.15% to about 0.45% |
| Citric Acid | From about 0.2% to about 0.6% |

In certain embodiments, the invention relates to any one of the aforementioned formulations, wherein the formulation comprises

| | |
|---|---|
| Hydrocortisone 17-butyrate | About 0.1% |
| Water | About 54% |
| Glycerine | About 5.0% |
| Methylparaben | About 0.3% |
| Propylparaben | About 0.1% |
| Cetostearyl Alcohol | About 5.3% |
| Urea | About 0.64% |
| Dimethicone | About 0.92% |
| White Petrolatum | About 6.9% |
| Mineral Oil | About 17.5% |
| Ceteth-20 | About 6.0% |
| Butylated hydroxytoluene | About 0.03% |
| Sodium Citrate | About 0.32% |
| Citric Acid | About 0.42% |

In certain embodiments, the invention relates to any one of the aforementioned formulations, wherein the formulation consists essentially of

| | |
|---|---|
| Hydrocortisone 17-butyrate | About 0.1% |
| Water | About 54% |
| Glycerine | About 5.0% |
| Methylparaben | About 0.3% |
| Propylparaben | About 0.1% |
| Cetostearyl Alcohol | About 5.3% |
| Urea | About 0.64% |
| Dimethicone | About 0.92% |
| White Petrolatum | About 6.9% |
| Mineral Oil | About 17.5% |
| Ceteth-20 | About 6.0% |
| Butylated hydroxytoluene | About 0.03% |
| Sodium Citrate | About 0.32% |
| Citric Acid | About 0.42% |

In certain embodiments, the invention relates to any one of the aforementioned formulations, wherein the formulation consists of

| | |
|---|---|
| Hydrocortisone 17-butyrate | About 0.1% |
| Water | About 54% |
| Glycerine | About 5.0% |
| Methylparaben | About 0.3% |
| Propylparaben | About 0.1% |
| Cetostearyl Alcohol | About 5.3% |
| Urea | About 0.64% |
| Dimethicone | About 0.92% |
| White Petrolatum | About 6.9% |
| Mineral Oil | About 17.5% |
| Ceteth-20 | About 6.0% |
| Butylated hydroxytoluene | About 0.03% |
| Sodium Citrate | About 0.32% |
| Citric Acid | About 0.42% |

In certain embodiments, the invention relates to any one of the aforementioned formulations, wherein the formulation comprises

Hydrocortisone 17-butyrate
Water
Glycerin
Methylparaben
Propylparaben
Cetostearyl Alcohol
Urea
Dimethicone
Vegetable Oil
White Petrolatum
Mineral Oil
Ceteth-20
Butylated hydroxytoluene
Sodium Citrate
Citric Acid

In certain embodiments, the invention relates to any one of the aforementioned formulations, wherein the formulation consists essentially of

Hydrocortisone 17-butyrate
Water
Glycerin
Methylparaben
Propylparaben
Cetostearyl Alcohol
Urea
Dimethicone
Vegetable Oil
White Petrolatum
Mineral Oil
Ceteth-20
Butylated hydroxytoluene
Sodium Citrate
Citric Acid

In certain embodiments, the invention relates to any one of the aforementioned formulations, wherein the formulation consists of

Hydrocortisone 17-butyrate
Water
Glycerin
Methylparaben
Propylparaben
Cetostearyl Alcohol
Urea
Dimethicone
Vegetable Oil
White Petrolatum
Mineral Oil
Ceteth-20
Butylated hydroxytoluene
Sodium Citrate
Citric Acid

In certain embodiments, the invention relates to any one of the aforementioned formulations, wherein the formulation comprises, by weight of the formulation

| | |
|---|---|
| Hydrocortisone 17-butyrate | From about 0.01% to about 0.2% |
| Water | From about 30% to about 86% |
| Glycerin | From about 2.5% to about 7.5% |
| Methylparaben | From about 0.15% to about 0.45% |
| Propylparaben | From about 0.05% to about 0.15% |
| Cetostearyl Alcohol | From about 2.0% to about 8.0% |
| Urea | From about 0.3% to about 0.9% |
| Dimethicone | From about 0.5% to about 1.5% |
| Vegetable oil | From about 6.0% to about 18.0% |
| White Petrolatum | From about 3.0% to about 9.0% |
| Mineral Oil | From about 2.0% to about 6.0% |
| Ceteth-20 | From about 3.0% to about 9.0% |
| Butylated hydroxytoluene | From about 0.01% to about 0.05% |
| Sodium Citrate | From about 0.15% to about 0.45% |
| Citric Acid | From about 0.2% to about 0.6% |

In certain embodiments, the invention relates to any one of the aforementioned formulations, wherein the formulation consists essentially of, by weight of the formulation

| | |
|---|---|
| Hydrocortisone 17-butyrate | From about 0.01% to about 0.2% |
| Water | From about 30% to about 86% |
| Glycerin | From about 2.5% to about 7.5% |
| Methylparaben | From about 0.15% to about 0.45% |
| Propylparaben | From about 0.05% to about 0.15% |
| Cetostearyl Alcohol | From about 2.0% to about 8.0% |
| Urea | From about 0.3% to about 0.9% |
| Dimethicone | From about 0.5% to about 1.5% |
| Vegetable oil | From about 6.0% to about 18.0% |
| White Petrolatum | From about 3.0% to about 9.0% |
| Mineral Oil | From about 2.0% to about 6.0% |
| Ceteth-20 | From about 3.0% to about 9.0% |
| Butylated hydroxytoluene | From about 0.01% to about 0.05% |
| Sodium Citrate | From about 0.15% to about 0.45% |
| Citric Acid | From about 0.2% to about 0.6% |

In certain embodiments, the invention relates to any one of the aforementioned formulations, wherein the formulation consists of, by weight of the formulation

| | |
|---|---|
| Hydrocortisone 17-butyrate | From about 0.01% to about 0.2% |
| Water | From about 30% to about 86% |
| Glycerin | From about 2.5% to about 7.5% |
| Methylparaben | From about 0.15% to about 0.45% |
| Propylparaben | From about 0.05% to about 0.15% |
| Cetostearyl Alcohol | From about 2.0% to about 8.0% |
| Urea | From about 0.3% to about 0.9% |
| Dimethicone | From about 0.5% to about 1.5% |
| Vegetable oil | From about 6.0% to about 18.0% |
| White Petrolatum | From about 3.0% to about 9.0% |
| Mineral Oil | From about 2.0% to about 6.0% |
| Ceteth-20 | From about 3.0% to about 9.0% |
| Butylated hydroxytoluene | From about 0.01% to about 0.05% |
| Sodium Citrate | From about 0.15% to about 0.45% |
| Citric Acid | From about 0.2% to about 0.6% |

In certain embodiments, the invention relates to any one of the aforementioned formulations, wherein the formulation comprises

| | |
|---|---|
| Hydrocortisone 17-butyrate | About 0.1% |
| Water | About 54% |
| Glycerine | About 5.0% |
| Methylparaben | About 0.3% |
| Propylparaben | About 0.1% |
| Cetostearyl Alcohol | About 5.3% |
| Urea | About 0.64% |
| Dimethicone | About 0.92% |
| Vegetable oil | About 12.6% |
| White Petrolatum | About 6.9% |
| Mineral Oil | About 4.9% |
| Ceteth-20 | About 6.0% |
| Butylated hydroxytoluene | About 0.03% |
| Sodium Citrate | About 0.32% |
| Citric Acid | About 0.42% |

In certain embodiments, the invention relates to any one of the aforementioned formulations, wherein the formulation consists essentially of

| | |
|---|---|
| Hydrocortisone 17-butyrate | About 0.1% |
| Water | About 54% |
| Glycerine | About 5.0% |
| Methylparaben | About 0.3% |
| Propylparaben | About 0.1% |
| Cetostearyl Alcohol | About 5.3% |
| Urea | About 0.64% |
| Dimethicone | About 0.92% |
| Vegetable oil | About 12.6% |
| White Petrolatum | About 6.9% |
| Mineral Oil | About 4.9% |
| Ceteth-20 | About 6.0% |
| Butylated hydroxytoluene | About 0.03% |
| Sodium Citrate | About 0.32% |
| Citric Acid | About 0.42% |

In certain embodiments, the invention relates to any one of the aforementioned formulations, wherein the formulation consists of

| | |
|---|---|
| Hydrocortisone 17-butyrate | About 0.1% |
| Water | About 54% |
| Glycerine | About 5.0% |
| Methylparaben | About 0.3% |
| Propylparaben | About 0.1% |
| Cetostearyl Alcohol | About 5.3% |
| Urea | About 0.64% |
| Dimethicone | About 0.92% |
| Vegetable oil | About 12.6% |
| White Petrolatum | About 6.9% |
| Mineral Oil | About 4.9% |
| Ceteth-20 | About 6.0% |
| Butylated hydroxytoluene | About 0.03% |
| Sodium Citrate | About 0.32% |
| Citric Acid | About 0.42% |

In certain embodiments, the invention relates to any one of the aforementioned formulations, wherein the formulation comprises, by weight of the formulation

| | |
|---|---|
| Hydrocortisone 17-butyrate | From about 0.01% to about 0.2% |
| Water | From about 30% to about 86% |
| Glycerin | From about 2.5% to about 7.5% |
| Methylparaben | From about 0.15% to about 0.45% |
| Propylparaben | From about 0.05% to about 0.15% |
| Cetostearyl Alcohol | From about 4.0% to about 12.0% |
| Urea | From about 0.3% to about 0.9% |
| Dimethicone | From about 0.5% to about 1.5% |
| Vegetable oil | From about 6.0% to about 18.0% |
| White Petrolatum | From about 3.0% to about 9.0% |
| Mineral Oil | From about 2.0% to about 6.0% |
| Ceteth-20 | From about 2.0% to about 6.0% |
| Butylated hydroxytoluene | From about 0.01% to about 0.05% |
| Sodium Citrate | From about 0.15% to about 0.45% |
| Citric Acid | From about 0.2% to about 0.6% |

In certain embodiments, the invention relates to any one of the aforementioned formulations, wherein the formulation consists essentially of, by weight of the formulation

| | |
|---|---|
| Hydrocortisone 17-butyrate | From about 0.01% to about 0.2% |
| Water | From about 30% to about 86% |
| Glycerin | From about 2.5% to about 7.5% |
| Methylparaben | From about 0.15% to about 0.45% |
| Propylparaben | From about 0.05% to about 0.15% |
| Cetostearyl Alcohol | From about 4.0% to about 12.0% |
| Urea | From about 0.3% to about 0.9% |
| Dimethicone | From about 0.5% to about 1.5% |
| Vegetable oil | From about 6.0% to about 18.0% |
| White Petrolatum | From about 3.0% to about 9.0% |
| Mineral Oil | From about 2.0% to about 6.0% |
| Ceteth-20 | From about 2.0% to about 6.0% |
| Butylated hydroxytoluene | From about 0.01% to about 0.05% |
| Sodium Citrate | From about 0.15% to about 0.45% |
| Citric Acid | From about 0.2% to about 0.6% |

In certain embodiments, the invention relates to any one of the aforementioned formulations, wherein the formulation consists of, by weight of the formulation

| | |
|---|---|
| Hydrocortisone 17-butyrate | From about 0.01% to about 0.2% |
| Water | From about 30% to about 86% |
| Glycerin | From about 2.5% to about 7.5% |
| Methylparaben | From about 0.15% to about 0.45% |
| Propylparaben | From about 0.05% to about 0.15% |
| Cetostearyl Alcohol | From about 4.0% to about 12.0% |
| Urea | From about 0.3% to about 0.9% |
| Dimethicone | From about 0.5% to about 1.5% |
| Vegetable oil | From about 6.0% to about 18.0% |
| White Petrolatum | From about 3.0% to about 9.0% |
| Mineral Oil | From about 2.0% to about 6.0% |
| Ceteth-20 | From about 2.0% to about 6.0% |
| Butylated hydroxytoluene | From about 0.01% to about 0.05% |
| Sodium Citrate | From about 0.15% to about 0.45% |
| Citric Acid | From about 0.2% to about 0.6% |

In certain embodiments, the invention relates to any one of the aforementioned formulations, wherein the formulation comprises

| | |
|---|---|
| Hydrocortisone 17-butyrate | About 0.1% |
| Water | About 54% |
| Glycerine | About 5.0% |
| Methylparaben | About 0.3% |
| Propylparaben | About 0.1% |
| Cetostearyl Alcohol | About 7.6% |
| Urea | About 0.64% |
| Dimethicone | About 0.92% |
| Vegetable oil | About 12.6% |
| White Petrolatum | About 6.9% |
| Mineral Oil | About 4.9% |
| Ceteth-20 | About 3.8% |
| Butylated hydroxytoluene | About 0.03% |
| Sodium Citrate | About 0.32% |
| Citric Acid | About 0.42% |

In certain embodiments, the invention relates to any one of the aforementioned formulations, wherein the formulation consists essentially of

| | |
|---|---|
| Hydrocortisone 17-butyrate | About 0.1% |
| Water | About 54% |
| Glycerine | About 5.0% |
| Methylparaben | About 0.3% |
| Propylparaben | About 0.1% |
| Cetostearyl Alcohol | About 7.6% |
| Urea | About 0.64% |
| Dimethicone | About 0.92% |
| Vegetable oil | About 12.6% |
| White Petrolatum | About 6.9% |
| Mineral Oil | About 4.9% |
| Ceteth-20 | About 3.8% |
| Butylated hydroxytoluene | About 0.03% |
| Sodium Citrate | About 0.32% |
| Citric Acid | About 0.42% |

In certain embodiments, the invention relates to any one of the aforementioned formulations, wherein the formulation consists of

| | |
|---|---|
| Hydrocortisone 17-butyrate | About 0.1% |
| Water | About 54% |
| Glycerine | About 5.0% |
| Methylparaben | About 0.3% |
| Propylparaben | About 0.1% |
| Cetostearyl Alcohol | About 7.6% |
| Urea | About 0.64% |
| Dimethicone | About 0.92% |
| Vegetable oil | About 12.6% |
| White Petrolatum | About 6.9% |
| Mineral Oil | About 4.9% |
| Ceteth-20 | About 3.8% |
| Butylated hydroxytoluene | About 0.03% |
| Sodium Citrate | About 0.32% |
| Citric Acid | About 0.42% |

### Exemplary Methods of the Invention

In certain embodiments, the invention relates to a method of optimizing the rate of release of an active agent from a topical formulation, comprising the step of varying the weight ratio of vegetable oil-to-mineral oil from about 0.0 to about 2.6, and varying the weight ratio of co-surfactant-to-surfactant from about 0.89 to about 2.0, thereby forming an improved active agent-containing topical formulation.

In certain embodiments, the invention relates to any one of the aforementioned methods, wherein the ratios are varied simultaneously.

In certain embodiments, the invention relates to a method of optimizing the rate of release of an active agent from a topical formulation, comprising the step of varying the weight ratio of vegetable oil-to-mineral oil from about 0.03 to about 1.00, thereby forming an improved active agent-containing topical formulation.

In certain embodiments, the invention relates to any one of the aforementioned methods, wherein the rate of release of the active agent from the improved active agent-containing formulation is less variable over time than the rate of release of the active agent from a reference formulation. In other words, in certain embodiments, the method is a method of steadying the rate of release of the active agent from the topical formulation. In certain embodiments, the invention relates to any one of the aforementioned methods, wherein the method is a method of steadying the rate of release of the active agent from the topical formulation by increasing the weight ratio of vegetable oil-to-mineral oil.

In certain embodiments, the invention relates to any one of the aforementioned methods, wherein the rate of release of the active agent from the improved active agent-containing topical formulation is greater over time than the rate of release of the active agent from a reference formulation.

In certain embodiments, the invention relates to any one of the aforementioned methods, wherein the weight ratio of vegetable oil-to-mineral oil is about 0.03, about 0.06, about 0.13, about 0.2, about 0.55, about 0.75, or about 1.00. In certain embodiments, the invention relates to any one of the aforementioned methods, wherein the weight ratio of vegetable oil-to-mineral oil is about 0.2 or about 0.55.

In certain embodiments, the invention relates to any one of the aforementioned methods, wherein the weight ratio of vegetable oil-to-mineral oil is about 0.0, about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, or about 2.6.

In certain embodiments, the invention relates to any one of the aforementioned methods, wherein the weight ratio of co-surfactant-to-surfactant is about 0.89, about 1.0, about 1.1, about 1.2, about 1.3, about 1.4, about 1.5, about 1.6, about 1.7, about 1.8, about 1.9, or about 2.0.

In certain embodiments, the invention relates to any one of the aforementioned methods, wherein the active agent is a corticosteroid.

In certain embodiments, the invention relates to any one of the aforementioned methods, wherein the active agent is hydrocortisone 17-butyrate.

In certain embodiments, the invention relates to any one of the aforementioned methods, wherein the improved active agent-containing topical formulation is any one of the aforementioned topical formulations.

In certain embodiments, the invention relates to any one of the aforementioned methods, wherein the vegetable oil comprises mono- and poly-unsaturated fatty acids.

In certain embodiments, the invention relates to any one of the aforementioned methods, wherein the vegetable oil comprises mono- and poly-unsaturated fatty acids with acyl chain lengths between about 4 and about 28 carbons.

In certain embodiments, the invention relates to any one of the aforementioned methods, wherein the vegetable oil comprises poly-unsaturated fatty acids in an amount from about 10% to about 78% of the number of fatty acids.

In certain embodiments, the invention relates to any one of the aforementioned methods, wherein the poly-unsaturated fatty acid is linoleic acid.

In certain embodiments, the invention relates to any one of the aforementioned methods, wherein the vegetable oil is safflower oil, sunflower oil, corn oil, sesame oil, peanut oil, canola oil, or olive oil.

In certain embodiments, the invention relates to any one of the aforementioned methods, wherein the vegetable oil is safflower oil.

In certain embodiments, the invention relates to any one of the aforementioned methods, wherein the vegetable oil has a viscosity from about 30 cP to about 50 cP at 35° C.

In certain embodiments, the invention relates to any one of the aforementioned methods, wherein the vegetable oil has a HLB value from about 6 to about 8. In certain embodiments, the invention relates to any one of the aforementioned methods, wherein the vegetable oil has a HLB value of 6, 7, or 8.

In certain embodiments, the invention relates to any one of the aforementioned methods, wherein the mineral oil is light mineral oil.

In certain embodiments, the invention relates to any one of the aforementioned methods, wherein the mineral oil has a viscosity from about 10 cP to about 20 cP at 35° C.

In certain embodiments, the invention relates to any one of the aforementioned methods, wherein the mineral oil has a HLB value from about 9 to about 11. In certain embodiments, the invention relates to any one of the aforementioned methods, wherein the mineral oil has a HLB value of 10.

In certain embodiments, the invention relates to any one of the aforementioned methods, wherein the formulation is an oil-in-water emulsion.

### Exemplary Properties of Formulations of the Invention

In certain embodiments, the invention relates to any one of the aforementioned formulations, wherein the formulation is a cream, a lotion, or a foam.

In certain embodiments, the invention relates to any one of the aforementioned formulations that, upon application to the skin of an affected subject, is non-irritating.

In certain embodiments, the invention relates to any one of the aforementioned formulations that, upon application to the skin of an affected subject, is well-tolerated.

In certain embodiments, the invention relates to any one of the aforementioned formulations that, upon application to the skin of an affected subject, is non-cytotoxic.

In certain embodiments, the invention relates to any one of the aforementioned formulations that, upon application to the skin of an affected subject, is weakly sensitizing. In certain embodiments, the invention relates to any one of the aforementioned formulations that, upon application to the skin of an affected subject, is non-sensitizing.

In certain embodiments, the invention relates to any one of the aforementioned formulations that, upon application to the skin of an affected subject, does not produce edema or erythema.

In certain embodiments, the invention relates to any one of the aforementioned formulations that, upon application to the skin of an affected subject, improves bioavailability of the active agent as compared to a reference formulation, wherein the reference formulation has a lower ratio of vegetable oil/mineral oil.

In certain embodiments, the invention relates to any one of the aforementioned formulations that, upon application to the skin of an affected subject, shows a steadier rate of release of the active agent over time as compared to a reference formulation, wherein the reference formulation has a lower ratio of vegetable oil/mineral oil.

In certain embodiments, the invention relates to any one of the aforementioned formulations that, upon application to the skin of an affected subject, releases a larger quantity of the active agent as compared to a reference formulation, wherein the reference formulation has a lower ratio of vegetable oil/mineral oil.

### Exemplary Formulations of the Invention for Particular Uses

In certain embodiments, the invention relates to any one of the formulations for use in the treatment of a skin disorder.

In certain embodiments, the invention relates to any one of the aforementioned formulations, wherein the skin disorder is a dermatosis.

In certain embodiments, the invention relates to any one of the aforementioned formulations, wherein the skin disorder is atopic dermatitis.

### Exemplary Methods of Use

In certain embodiments, the invention relates to a method of treating a skin disorder, comprising the steps of:

applying topically to an area of skin of a subject in need thereof a therapeutically-effective amount of any one of the aforementioned formulations.

In certain embodiments, the invention relates to any one of the aforementioned methods, wherein the formulation is applied once daily or twice daily.

In certain embodiments, the invention relates to any one of the aforementioned methods, wherein the subject is human.

In certain embodiments, the invention relates to any one of the aforementioned methods, wherein the skin disorder is a dermatosis.

In certain embodiments, the invention relates to any one of the aforementioned methods, wherein the skin disorder is atopic dermatitis.

### EXEMPLIFICATION

The invention now being generally described, it will be more readily understood by reference to the following examples which are included merely for purposes of illustration of certain aspects and embodiments of the present invention, and are not intended to limit the invention.

### Example 1

### Compositions and Manufacturing Process of the Method

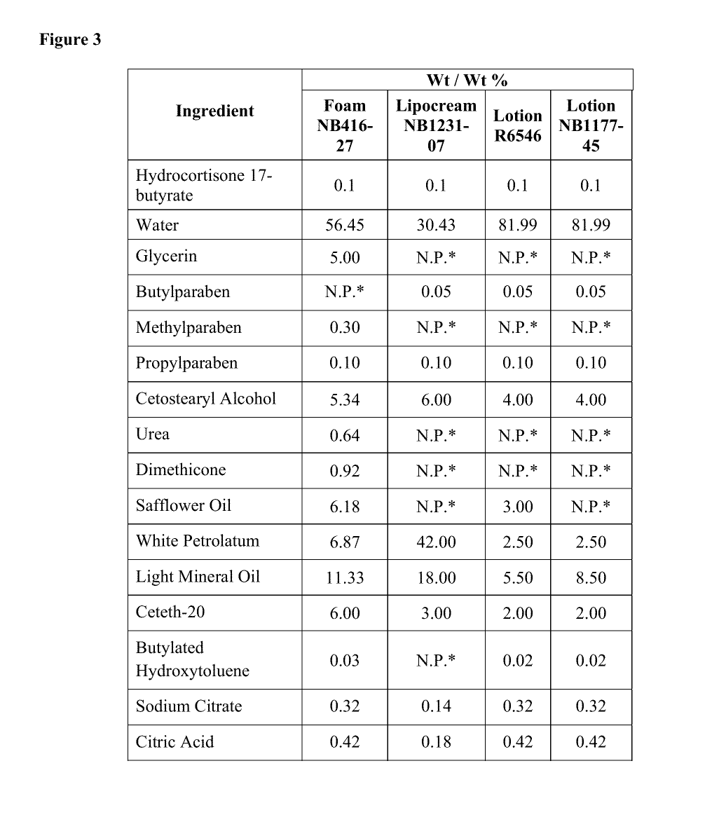

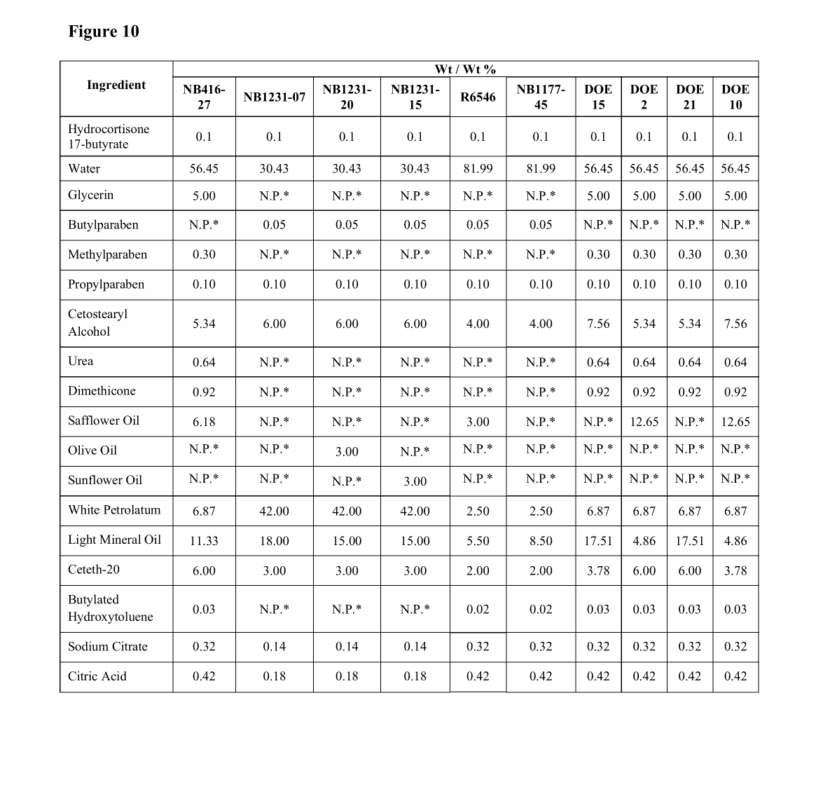

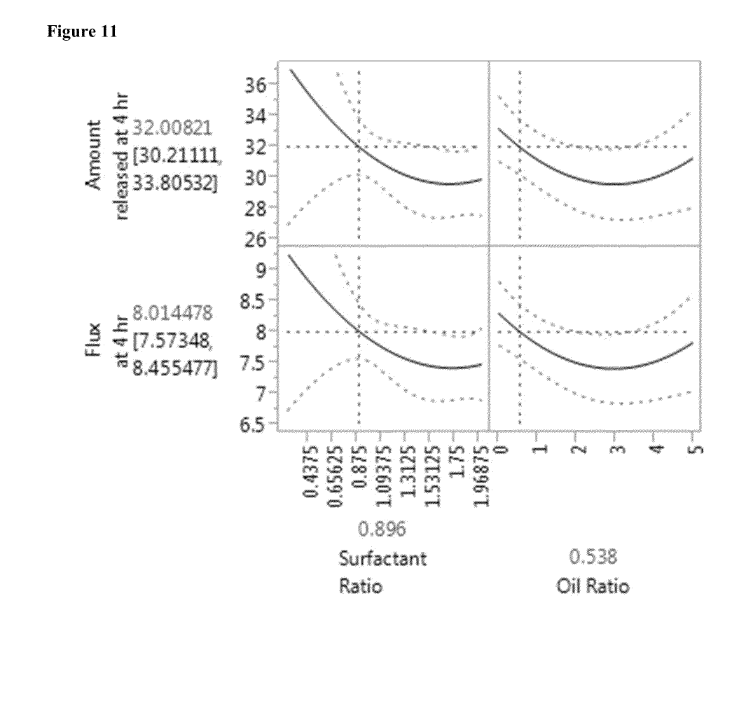

An example product concentrate (NB416-27; see FIG. 3 and FIG. 10) can be manufactured by the procedure outlined below:

### Part A: Oil Phase Preparation

1. Charge Ceteth-20 (I) light mineral oil, white petrolatum, dimethicone, safflower oil, butylated hydroxytoluene and cetostearyl alcohol into a Stainless Steel tank and heat until fully melted

### Part B: Aqueous Phase Preparation

1. Charge Purified Water (I) and Glycerin into a Stainless Steel tank and heat to 75-80° C.
2. Charge and dissolve citric acid (I) and sodium citrate (I) as well as urea, methyl paraben and propyl paraben while mixing.
3. Continue mixing until a clear solution is obtained while maintaining a temperature of 65-95° C.

### Part C: Drug Phase Preparation

1. Charge a Stainless Steel tank with Purified Water (II), citric acid (II), sodium citrate (II) and ceteth-20 (II).
2. Mix slowly at room temperature to dissolve.
3. Add hydrocortisone butyrate and mix until fully wetted and dispersed.

### Part D: Drug Product Concentrate Formation

1. Add Part A to Part B while high shear mixing at 65-95° C.
2. Cool the emulsion with an outside cold water jacket to below 50° C. while high shear mixing.
3. Discontinue high shear mixing. Start low shear mixing and continue cooling with cold water jacket to form the vehicle emulsion.
4. When the temperature of the vehicle emulsion is below 37° C., add Part C and continue mixing until uniform.
5. Cool to room temperature. Adjust to final volume with DI water. Mix until uniform.

Following manufacturing of the Drug Product and Vehicle Concentrate, the finished Drug Product and Drug Product Vehicle is produced as outlined below.
1. Aerosol cans are cleaned with compressed air and vacuum.
2. Product Concentrate is filled into cans.
3. Valves are placed onto the cans.
4. Cans are crimped and hydrofluorocarbon propellant is charged.
5. The aerosol can valve and dip-tube is purged with argon gas.

Propellant concentrations range from 8-15% by weight of packaged product, argon concentrations range from 0.8-4.0% by weight of packaged product.

### Example 2

### In Vitro Release Kinetics

In order for topically applied drug products to be effective, the drug substance must be released from the vehicle before it can traverse to the stratum corneum. Although not directly correlated to in vivo bioavailability, characterization of drug product release profiles allows for the identification of formulations with the potential to increase drug product bioavailability. A Franz vertical diffusion cell was used to examine the rate and extent of API release from foam concentrates in vitro. The experimental conditions were as below.
Instrument: Logan Instruments Corp System 912-12
Membrane: Whatman, PTFE, 5.0 um, 37 mm
Temperature: 32.5° C.
Speed: 300 rpm
Time pulls (min): 30, 60, 120, 240, and 360
Media: For Base Line Conditions: 70% Buffer, 30% Ethanol
1) Turn all parts of the instrument.
2) Prime, fill and drain sample cells three times
3) Fill the media reservoir with the appropriate media and repeat step 2.
4) Prepare the cells:
   a. Place the membrane on top of the cell, place the cap, and then clap them down together.
   b. Fill cells with media
   c. Transfer the sample via direct transfer. Make sure to take an initial weight and a final weight after filling every test article.
5) Collect samples
   a. Set flush volume to 1.5 mL
   b. Set media replace volume to 4.6 mL with return to cell
   c. Set waste to 1.5 mL
   d. Set sample to 1.5 mL
   e. Set sampling time intervals
6) Measure hydrocortisone butyrate concentration in samples via HPLC
   Instrument: Liquid Chromatograph equipped with a UV Detector
   Column: Zorbax™ SB-CN Dimensions: 150×4.6 mm, 3.5 μm, Agilent® Part Number 863953-905 or equivalent
   Mobile Phase A Composition: 5 mM Phosphate Buffer pH4.8
   Mobile Phase B Composition: Methanol
   Mobile Phase C Composition: Acetonitrile

Mobile Phase Composition Table:

| Mobile | Mobile | Mobile |
|---|---|---|
| Phase A | Phase B | Phase C |
| % | % | % |
| 60 | 20 | 20 |

Column Temperature: 40° C.
Flow Rate: 1.2 mL/min
Detection: UV at 245 nm
Injection Volume: 25 μL
Run Time: 20 min

1) Sample Preparation

I. Foam Sample:
a. Load autosampler with HPLC sample vials
b. Fill chambers with
c. Dispense 10-15 grams drug product concentrate
d. Fill a syringe with sample.
e. Tare a balance, place the syringe containing the sample on the balance, and record the weight.
f. Slowly add approximately 0.8-1.0 g of sample into cell chamber #1 (ensure that the sample fills the cell chamber, avoid creating air gaps or headspace between the sample and filter).
g. Place the syringe back onto the tared balance and record the weight in grams, record the weight (Back weighing).

Continue with steps g through h until all sample cell chambers are full.

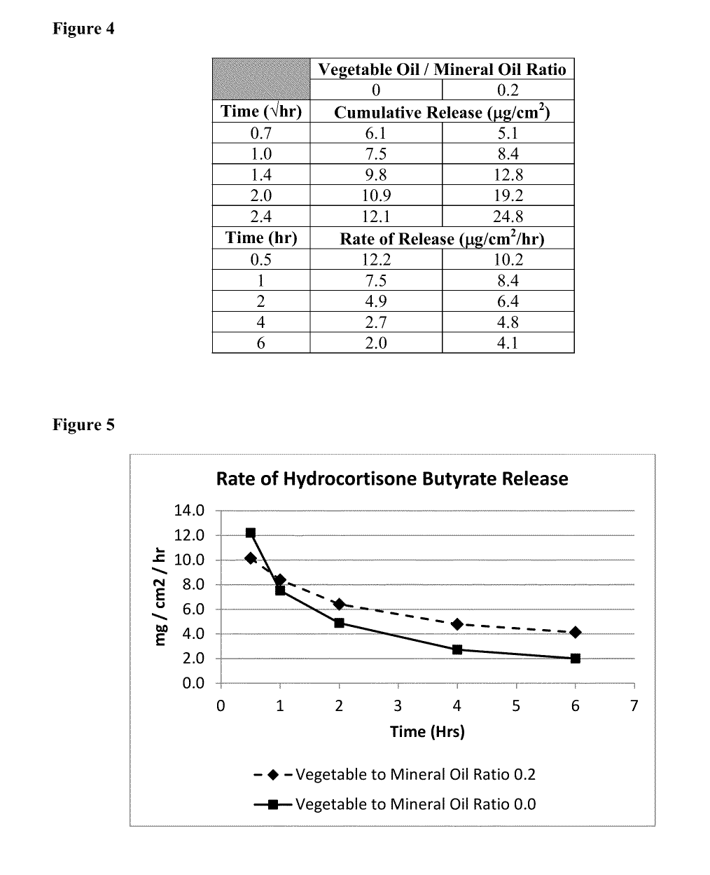

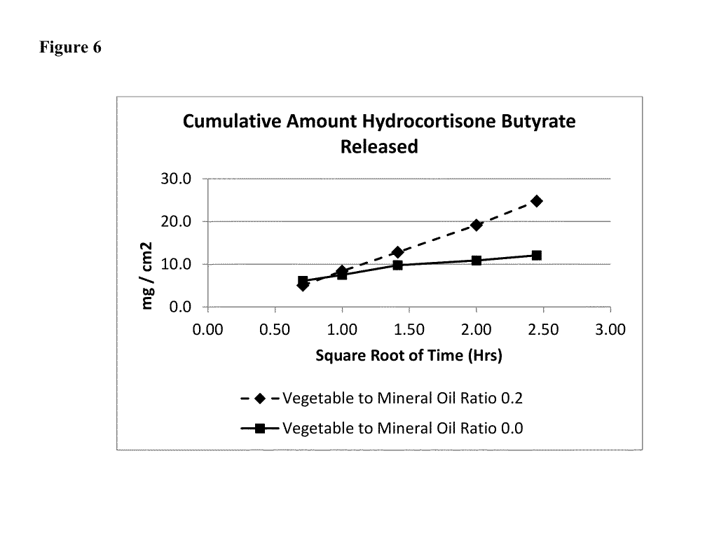

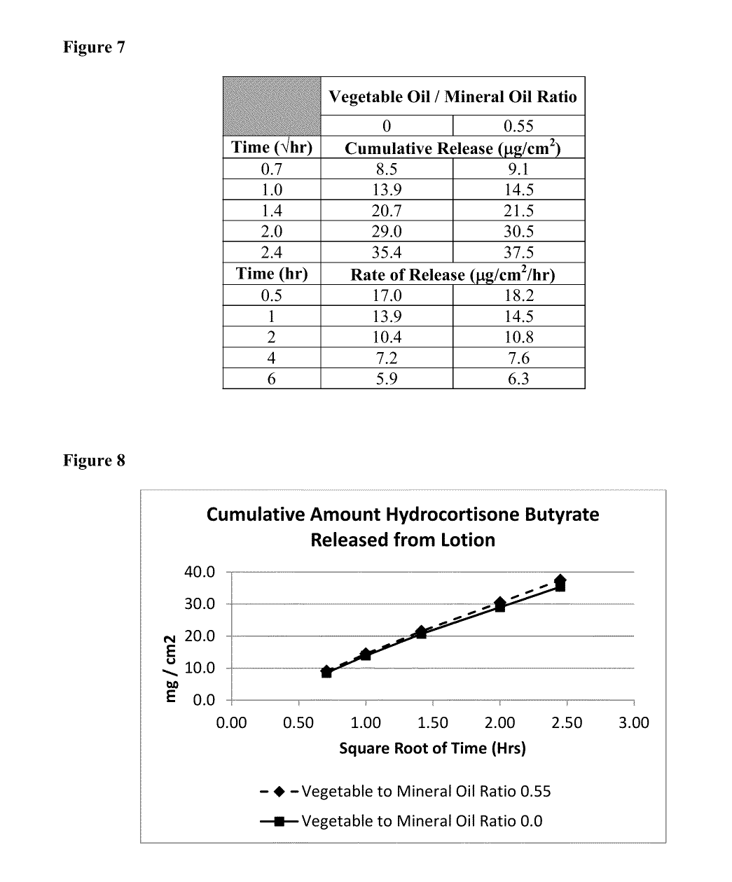

See FIGS. 4-8 and 11-13.

### Example 3

### Determination of the Effect of Varied Vegetable Oils on In Vitro Release Kinetics

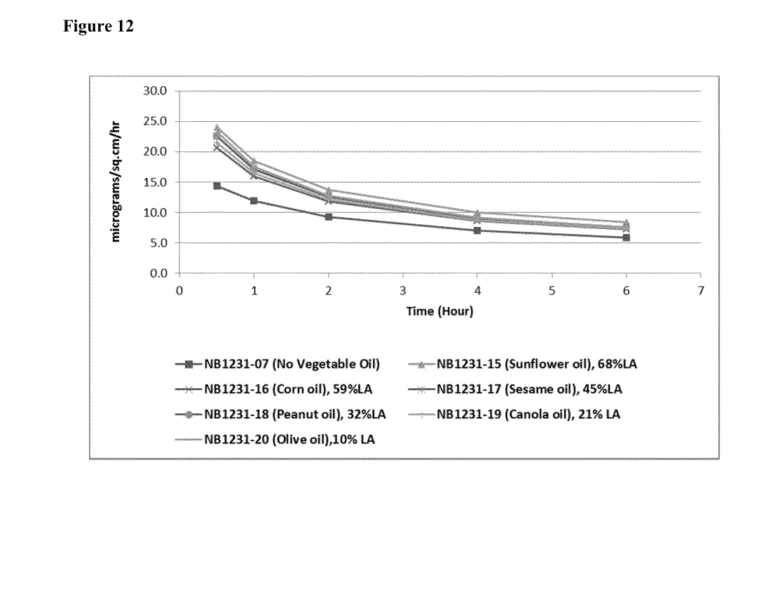

In order to demonstrate that the release rate modification observed in the presence of vegetable oil was not oil-specific a series of exemplary formulations were prepared as per Example 1 and tested for in vitro release as described in Example 2. As shown in FIG. 5 and FIG. 12, addition of vegetable oil to the exemplary formulations increased the in vitro rate of active release compared to formulations containing solely mineral oil.

### Example 4

### Optimization of Vegetable/Mineral Oil and Co-Surfactant/Surfactant Ratios

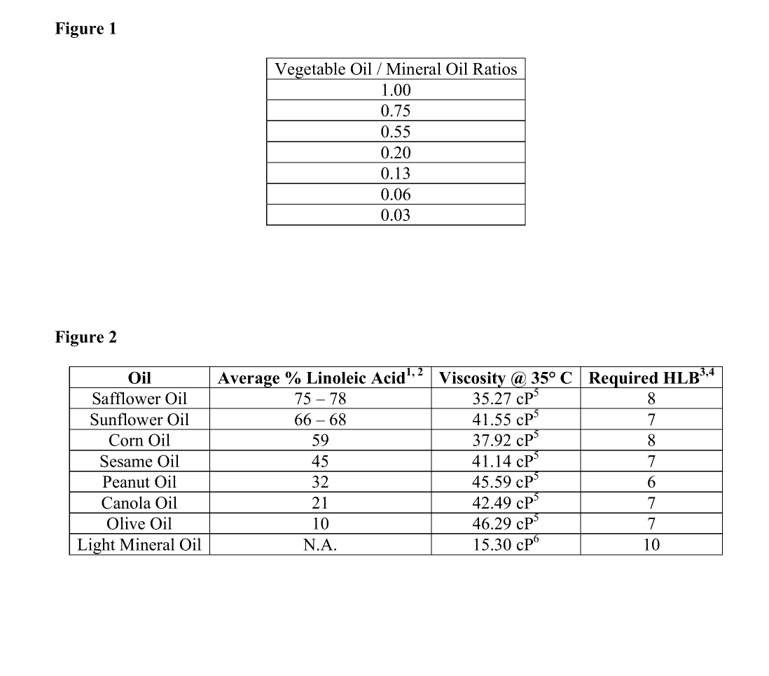

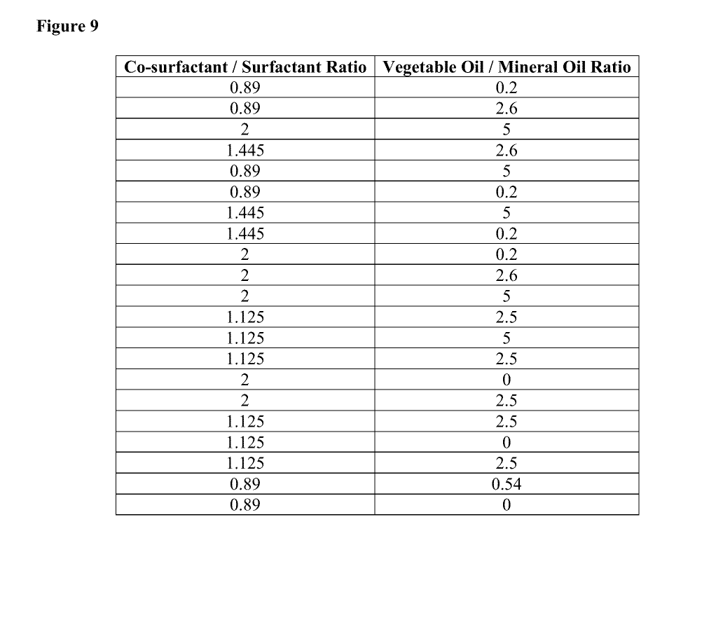

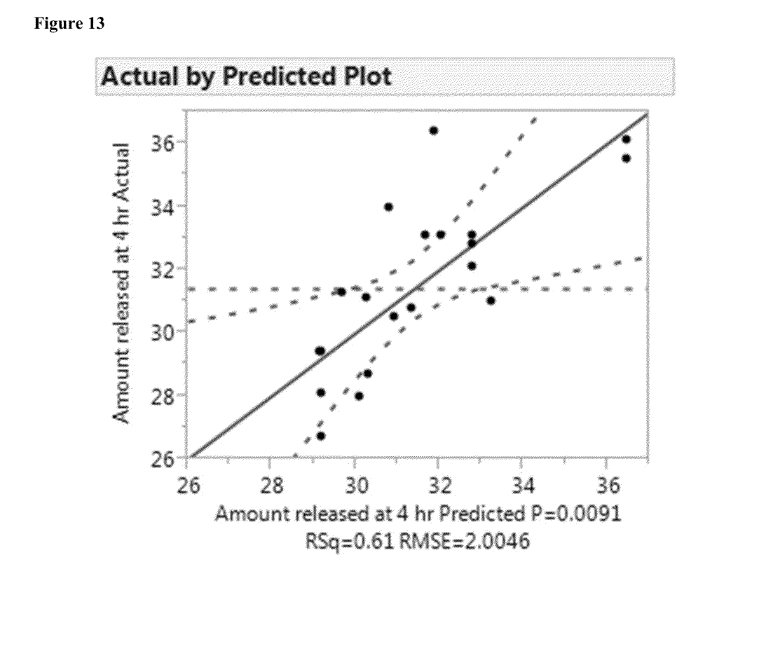

In order to develop a mathematical model for the optimization of in vitro release kinetics, a series of exemplary formulations containing different ratios of vegetable/mineral oil and co-surfactant/surfactant were prepared (FIG. 1 and FIG. 9) as described in Example 1 and tested for in vitro release as described in Example 2. As shown in FIG. 11, the rate of in vitro release is simultaneously dependent on the ratio of vegetable/mineral oil and co-surfactant/surfactant such that simultaneous modification of both ratios is required to achieve a given in vitro release profile. FIG. 13 shows the agreement between theoretical and experimental in vitro release kinetics for exemplary formulations with the vegetable/mineral oil and co-surfactant/surfactant ratios given in FIG. 9.

### INCORPORATION BY REFERENCE

All of the U.S. patents and U.S. published patent applications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A method of optimizing the rate of release of an active agent from a topical formulation, comprising the step of varying the weight ratio of vegetable oil-to-mineral oil from about 0.0 to about 2.6, and varying the weight ratio of co-surfactant-to-surfactant from about 0.89 to about 2.0, thereby forming an improved active agent-containing topical formulation.

2. The method of claim 1, wherein the rate of release of the active agent from the improved active agent-containing topical formulation is greater over time than the rate of release of the active agent from a reference formulation.

3. The method of claim 1, wherein the weight ratio of vegetable oil-to-mineral oil in the improved active agent-containing formulation is about 0, about 0.03, about 0.06, about 0.13, about 0.2, about 0.55, about 0.75, about 1.0, about 1.25, about 1.50, about 1.75, about 2.0, about 2.25, or about 2.6; and the weight ratio of co-surfactant-to-surfactant in the improved active agent-containing formulation is about 0.89, about 1.0, about 1.1, about 1.2, about 1.3, about 1.4, about 1.5, about 1.6, about 1.7, about 1.8, about 1.9, or about 2.0.

4. The method of claim 1, wherein the active agent is a corticosteroid.

5. The method of claim 1, wherein the active agent is hydrocortisone 17-butyrate.

6. The method of claim 1, wherein the improved active agent-containing topical formulation comprises vegetable oil; and the vegetable oil comprises poly-unsaturated fatty acids in an amount from about 10% to about 78% of the number of fatty acids.

7. The method of claim 6, wherein the poly-unsaturated fatty acid is linoleic acid.

8. The method of claim 1, wherein the improved active agent-containing topical formulation comprises vegetable oil; and the vegetable oil is safflower oil, sunflower oil, corn oil, sesame oil, peanut oil, canola oil, or olive oil.

9. The method of claim 1, wherein the improved active agent-containing topical formulation comprises vegetable oil; and the vegetable oil is safflower oil.

10. The method of claim 1, wherein the mineral oil is light mineral oil.

11. The method of claim 1, wherein the improved active agent-containing topical formulation is an oil-in-water emulsion.

12. The method of claim 1, wherein the improved active agent-containing topical formulation consists essentially of, by weight of the formulation

| | |
|---|---|
| Hydrocortisone 17-butyrate | From about 0.01% to about 0.2% |
| Water | From about 20% to about 90% |
| Propylparaben | From about 0.05% to about 0.15% |
| Cetostearyl Alcohol | From about 2% to about 9% |
| White Petrolatum | From about 1.5% to about 60% |
| Ceteth-20 | From about 1% to about 9% |
| Sodium Citrate | From about 0.07% to about 0.50% |
| Citric Acid | From about 0.09% to about 0.60% |
| Vegetable oil | From about 1% to about 9% |
| Mineral oil | From about 2% to about 27% |

13. The method of claim 1, wherein the improved active agent-containing topical formulation consists essentially of, by weight of the formulation

| | |
|---|---|
| Hydrocortisone 17-butyrate | From about 0.01% to about 0.2% |
| Water | From about 40% to about 70% |
| Glycerin | From about 2% to about 8% |
| Methylparaben | From about 0.1% to about 0.5% |
| Propylparaben | From about 0.05% to about 0.15% |
| Cetostearyl Alcohol | From about 2% to about 8% |
| Urea | From about 0.3% to about 0.9% |
| Dimethicone | From about 0.5% to about 1.5% |
| Vegetable oil | From about 3% to about 9% |
| White Petrolatum | From about 3% to about 9% |
| Mineral Oil | From about 6% to about 18% |
| Ceteth-20 | From about 3% to about 9% |
| Butylated Hydroxytoluene | From about 0.015% to about 0.045% |
| Sodium Citrate | From about 0.015% to about 0.045% |
| Citric Acid | From about 0.2% to about 0.6% |

14. The method of claim 1, wherein the improved active agent-containing topical formulation consists essentially of, by weight of the formulation,

| | |
|---|---|
| Hydrocortisone 17-butyrate | From about 0.01% to about 0.2% |
| Water | From about 15% to about 45% |
| Butylparaben | From about 0.02% to about 0.08% |
| Propylparaben | From about 0.05% to about 0.15% |
| Cetostearyl Alcohol | From about 3% to about 9% |
| Vegetable Oil | From about 1.5% to about 4.5% |
| White Petrolatum | From about 20% to about 60% |
| Mineral Oil | From about 8% to about 22% |
| Ceteth-20 | From about 1.5% to about 4.5% |
| Sodium Citrate | From about 0.06% to about 0.22% |
| Citric Acid | From about 0.09% to about 0.27% |

15. The method of claim 1, wherein the improved active agent-containing topical formulation consists essentially of, by weight of the formulation

| | |
|---|---|
| Hydrocortisone 17-butyrate | From about 0.01% to about 0.2% |
| Water | From about 45% to about 95% |
| Butylparaben | From about 0.02% to about 0.08% |
| Propylparaben | From about 0.05% to about 0.15% |
| Cetostearyl Alcohol | From about 2% to about 6% |
| vegetable Oil | From about 1.5% to about 4.5% |
| White Petrolatum | From about 1% to about 5% |
| Mineral Oil | From about 2% to about 8% |
| Ceteth-20 | From about 1% to about 3% |
| Butylated Hydroxytoluene | From about 0.01% to about 0.03% |
| Sodium Citrate | From about 0.15% to about 0.45% |
| Citric Acid | From about 0.2% to about 0.6% |

16. A method of treating a skin disorder, comprising the steps of:
applying topically to an area of skin of a subject in need thereof a therapeutically-effective amount of an improved active agent-containing topical formulation of claim 1.

17. The method of claim 16, wherein the formulation is applied once daily or twice daily.

18. The method of claim 16, wherein the subject is human.

19. The method of claim 16, wherein the skin disorder is a dermatosis.

20. The method of claim 16, wherein the skin disorder is atopic dermatitis.
